(19) United States
(12) Patent Application Publication
(10) Pub. No.: US 2012/0058542 A1
(43) Pub. Date: Mar. 8, 2012

(54) SYSTEMS AND METHODS FOR REGULATING ALGAL BIOMASS

(75) Inventors: Benjamin Chiau-pin Wu, San Ramon, CA (US); David Stephen, Davis, CA (US); Gaye Elizabeth Morgenthaler, Woodside, CA (US); David Vancott Jones, Woodside, CA (US)

(73) Assignee: Live Fuels ,Inc., Menlo Park, CA (US)

(21) Appl. No.: 13/203,827

(22) PCT Filed: Mar. 10, 2010
(86) PCT No.: PCT/US2010/026761

§ 371 Date: Nov. 10, 2011

Related U.S. Application Data
(60) Provisional application No. 61/159,367, filed on Mar. 11, 2009.

(51) Int. Cl. C12N 1/12; A01G 1/00; A01K 61/00; C02F 3/32; C10L 1/00; A23L 1/326

(52) U.S. Cl. 435/257.1; 44/307; 426/643; 119/230; 119/200; 210/602; 210/170.09; 210/85; 210/170.05; 47/1.4

(57) ABSTRACT

The invention relates to systems and methods for regulating algal biomass in offshore waters near an oil and gas production platform. The systems of the invention encompasses a plurality of modules for managing nutrients, algae, and aquaculture, including enclosures for containing aquatic organisms, and various operating subsystems that are operably associated with surface and underwater structures of the platform. In one embodiment of the invention, aquatic organisms are cultured in eutrophic water to feed on algae, thereby reducing the algal biomass. In other embodiments, the diversity of algae in an algal bloom is modified and the productivity of oligotrophic water is increased.

### 1. INTRODUCTION

The invention relates to systems and methods for regulating algal biomass offshore, particularly in a body of water proximate to a hydrocarbon production installation.

### 2. BACKGROUND OF THE INVENTION

Recent coastal surveys of Unites States and Europe found that 78% of the assessed continental US coasts and 65% of Europe's Atlantic coast exhibit symptoms of nutrient over-enrichment (Bricker et al., 2007, Effects of Nutrient Enrichment in the Nation's Estuaries: A Decade of Change. NOAA Coastal Ocean Program Decision Analysis Series No. 26. Silver Spring, Md.: National Centers for Coastal Ocean Science; OSPAR integrated report 2003 on the eutrophication status. OSPAR Commission, 2003, London, U.K). The major nutrients that cause over-enrichment are nitrogen and phosphorus. The sources of nutrients vary among regions and are very often remote from the body of affected water. Agriculture, human sewage, urban run-off, industrial effluent, and fossil fuel combustions are the most common sources. The intensive use of commercial fertilizers over the last 50 years has increased crop yield but much of the nutrients not absorbed by crops find their way into local rivers and lakes, and are carried downstream to the estuaries, and eventually to the sea. Human activities have resulted in the near doubling of nitrogen and tripling of phosphorus flows to the environment when compared to natural causes. Trends in agricultural practices, energy use, and population growth indicate that eutrophication, especially in coastal areas, will be an ever-growing problem.

Nutrient over-enrichment often leads to algal bloom resulting in changes in biodiversity and loss of habitats (e.g., decline of coral reef), and appearance of “dead zones” where most animals are driven away or die of hypoxia. Where excess algal proliferation is dominated by certain species of toxic algae, mass mortalities of wild fish and shellfish, birds and even mammals occur; fish farms that use these waters are also impacted. Paralysis, diarrhea, and amnesia were associated with human consumption of contaminated seafood. Change in abundance and diversity of living organisms have adverse impacts on the environmental quality of coastal waters. Of the 415 areas around the world identified as experiencing some form of eutrophication, 169 are hypoxic (Selman et al., Eutrophication and hypoxia in coastal areas: a global assessment of the state of knowledge. World Resource Institute Policy Note, March 2008.) The Gulf of Mexico has a seasonal hypoxic zone that forms every year in late summer. Its size varies, in 2000, it was less than 5,000 km², while in 2002, it was approximately 22,000 km² (or the size of the state of Massachusetts). There is an urgent need to abate the deterioration of the quality of water in the world's coastal regions.

There are more than 6,500 offshore oil and gas installations around the world, in some 53 countries. The main areas are Gulf of Mexico: 4,000, Asia: 950, Middle East: 700, North Sea and North East Atlantic: 490, West Africa coast: 380, and South America: 34. The Gulf of Mexico is the most extensively developed and mature offshore petroleum-producing region in the world. In the federally regulated outer continental shelf of the Gulf of Mexico, 40,000 wells have been drilled and nearly 33,000 miles of pipeline are currently in use. When the time arrives that the cost to operate an offshore structure exceeds the income from the hydrocarbons under production, the structure exists as a liability and becomes a candidate for divestiture or decommissioning (Kaiser and Iledare, 2006, Journal of Construction Engineering and Management, 132:815-826). In the course of erecting and operating the structures, the aquatic environment surrounding the structure is subject to pollution by heavy metal and oil leakage. Federal regulations require that all structures be removed and all wells plugged and abandoned within one year the subsea lease is terminated. Since 1947 when offshore production began, over 2,200 structures have been removed, and over the last decade, 125 structures on average have been decommissioned annually. Based on data between 1998-2003, the average cost to remove a structure was estimated to be $1,000 per ton. For example, it has been estimated that the cost to remove to shore an 8-pile, 4-well, fixed platform in the Central Gulf of Mexico in 210 feet water depth with a total weight of 980 ton and two pipeline connections would be $1.1 million. If the structure is donated to a reef program, the expected cost would be $831,000 (Kaiser, 2006, Journal of Construction Engineering and Management, 132: 249-258). Moreover, oil and gas platforms located in eutrophic waters with harmful algal bloom or hypoxia are not suitable for fish farming by intensive monoculture or for developing artificial reef or angling resort.

Citation of any reference in Section 2 of this application is not to be construed as an admission that such reference is prior art to the present application.

### 3. SUMMARY OF THE INVENTION

The invention provides biological methods for regulating algal biomass in a body of eutrophic water comprising culturing organisms that feed on the algae. The organisms used in the invention are fishes and/or shellfishes. Also provided are oil and gas platform-based systems for managing nutrients, algae and aquatic organisms. It is contemplated that a network of such platform-based systems can remediate the aquatic environment of a wide geographic area.

The invention is particularly applicable in eutrophic water found offshore in areas where structures for hydrocarbon production are located. Typically, the sources of the nutrients in eutrophic water are agricultural runoff, industrial effluent, sewage, upwelling, aquaculture waste, or a mixture thereof. The oversupply of nutrients in a body of eutrophic water support algal growth leading to an increase in algae biomass, an algal bloom, a harmful algal bloom, or hypoxia. Eutrophic water and algal bloom can be identified by one or more techniques, such as but not limited to analysis of algae in water samples and remote sensing. Optionally, the data can be analyzed in combination with historical data for a region. For example, eutrophic water can be found in a location where an algal bloom has occurred previously or occurs on a regular, diurnal, seasonal, or annual basis.

A community of different algae comprising microalgae such as raphidophytes, dinoflagellates, diatoms, and/or cyanobacteria are found in eutrophic water. In an algal bloom, bloom species belonging to the following taxonmic groups are found: Anabaena, Aphanizomenon, Microcystis, Noctiluca, Alexandrium, Prorocentrum, Gymnodinium, Ceratium, Pfiesteria, Dinophysis, Gyrodinium, Heterosigma, Chattonella, Skeletonema, Synedropsis, Pseudonitzschia, Leptocylindrus, Chaetoceros; Phaeocystis, Nannochloris, Stichococcus, Aureococcus, Miraltia, Guinardia, Spermatozopsis, Urosolenia, Nitschia, Cyclotella, Cryptomonas, and Pedinophora. The sizes of the algae including bloom species can range from about 20-200 μm, about 2-20 μm, or about 0.2-2 μm.

Depending on the bloom species and water conditions, such as turbidity, dissolved oxygen level and toxin level, the invention contemplates using fishes and/or shellfishes of various sizes, ages, or developmental forms to consume the algae. Planktivorous organisms that have developed specialized structures (e.g., gill rakers in fish such as menhaden, or gill lamellae in bivalves such as green mussels) to feed on plankton, phytoplankton in particular, are preferred. The planktivorous organisms in the enclosure can be chosen for their preference for plankton of a particular size range that matches the size of the bloom species. As the composition and size distribution of the plankton community in a bloom changes over time, a different assemblage of planktivorous organisms with appropriately matching preferences of food size or type can be used.

In preferred embodiments, the planktivorous organisms are fishes of the order Clupiformes, which include but is not limited to, menhaden, anchovies, shads, sardines, pilchards, herring, and hilsas. Preferably, gulf menhaden or Atlantic menhaden are used in the Gulf of Mexico and east coasts of North America respectively. The shellfishes used in the methods of the invention are preferably bivalves, such as but not limited to oysters, mussels, scallops, and clams. Depending on the local environment, marine and/or brackish species can be used.

In various embodiments, the planktivorous organisms are introduced into an enclosure situated in a body of eutrophic water. The water can be populated with the organisms before the start of a regular algal bloom, or after a bloom has developed. By manipulating the timing of sexual maturation, mating, and spawning of stocks, planktivorous organisms of a certain age or developmental stage can be produced for use in the enclosure at any time during the year. The fishes and shellfishes are cultured within the enclosure of the invention to consume the algae until the algal biomass is reduced or when the fishes and shellfishes have grown to a certain size suitable for harvesting and processing. The enclosure can be restocked with juveniles of the organisms after harvesting. The planktivorous organisms are harvested or gathered and processed by methods known in the art for seafood processing and rendering of fish meal and fish oil. The fish oil can be used as an energy feedstock. Shellfishes can be harvested for human consumption. Methods for making biofuel from fishes grown in enclosures and harvested from the eutrophic water, are aspects of the invention.

In another embodiment, the invention provides a method for modifying the diversity of algal species in a body of water comprising a growing algal population that has more than 10⁵ algal cells per ml. This method can be useful to avoid the development of a harmful algal bloom. It is apparent that the growth rates of certain species of algae in water are limited by the concentration of certain macronutrients and/or micronutrients. A stream of fluid comprising a macronutrient and/or a micronutrient is transported from a distance or a different depth by hydrocarbon production installations, such as pipelines, and discharged into a body of water with a growing algal population proximate to a hydrocarbon production installation. A change in concentrations of macronutrients and/or micronutrients in the ambient water will influence the relative growth rates of various algae species, and limit the dominance of toxic algal species. The stream of fluid can be iron-enriched seawater.

In yet another embodiment, the invention provides a method for increasing the productivity of a body of oligotrophic water. The method comprises transporting a stream of eutrophic water comprising a macronutrient and/or a micronutrient by hydrocarbon production installations, such as pipelines, and discharging the stream of eutrophic water into the body of oligotrophic water. The stream of water taken from a distance or a different depth comprises a higher concentration of the macronutrient and/or micronutrient relative to the body of oligotrophic water. The increase in macronutrients and/or micronutrients will stimulate the growth of algae and other aquatic organisms in the body of water.

Also encompassed in the invention are oil and gas platform-based systems for regulating algal biomass. Such systems comprise a plurality of modules for managing nutrients, algae, and aquaculture that surround a hydrocarbon production installation. These modules are operably associated with the structural members of hydrocarbon production installations including platforms, subsea structures and pipelines. The nutrient management module can comprise pipelines and/or risers that are fluidically connected with a plurality of hydrocarbon production installations, including platforms and subsea structures that are horizontally spaced in a geographic area. The pipelines and risers can be connected to a variety of hydrocarbon production installations that are vertically disposed below, or on the seabed, at various water depths, or above water up to the height of a platform deck. The algae module comprises satellite, aerial, and/or in situ imaging subsystems for measuring algae density, counting algae and/or classifying algae taxonomically, and data subsystems for analysis, storage, and display of algal biomass data. The aquaculture module comprise one or more enclosures, and an operating subsystem which can comprise an environmental monitoring subsystem, a feeding subsystem, an aeration subsystem, a transfer subsystem, and a fish processing facility.

### 4. BRIEF DESCRIPTION OF THE DRAWING

FIG. 1 shows a framework for the application of planktivorous-trophic level bioremediation technology. Box 1 indicates an embodiment of the invention directed to reducing algal biomass in eutrophic coastal waters (marine and brackish ecosystems). Box 2 indicates an embodiment of the invention that is applicable to inland waters. Box 3 indicates an embodiment of the invention useful for reducing algal biomass in waste water discharged from, for example, industrial plants, animal husbandry operations, or fish farms.

FIG. 2 shows the cross-section of the substructure of a platform that has 16 upright members, and a production riser 101. Four of the upright members 102 are located inside an area defined by the twelve outer or peripheral upright members 103. The dashed lines 104 indicate mesh that forms the side walls of the two enclosures: an inner enclosure 105 and an outer enclosure 106. The top and bottom walls of the enclosures, horizontal, diagonal, and traverse members are not shown.

FIG. 3 is a simplified schematic of a platform 200 with its superstructure 210 situated above sea level 201. Upright members 203 secured to the seabed 202 by piles 204 are braced by horizontal members 205 and diagonal members 206. Three exemplary types of enclosures 208, 212, 230 are shown. Meshed panels 207 are mounted onto the upright and horizontal members to form the side walls of an enclosure 208. The top and bottom walls of the enclosure are not shown. Another enclosure 212 is defined by peripheral upright members 217, frame members 214, and a guy wire 213 which is anchored to the seabed by a pile 215. The walls of the enclosure 212 is formed by mesh 211 attached to the guy wires, upright members and the frame members. Another guy wire 218 that is attached to the superstructure can be used to support a subsystem or form another enclosure. Enclosure 230 is formed by a cage 231 rigidly or flexibly connected to an upright member via an attachment assembly 232.

### 5. DETAILED DESCRIPTION OF THE INVENTION

The invention relates to systems and methods for regulating algal biomass in offshore areas where hydrocarbon production installations are located. In one embodiment, the invention provides a system comprising a hydrocarbon production installation; a body of eutrophic water containing algae proximate to the hydrocarbon production installation; and a plurality of organisms that feed on algae in the body of eutrophic water. The methods of the invention for reducing algal biomass in eutrophic water comprise populating a body of eutrophic water that is proximate to a hydrocarbon production installation with a plurality of organisms that feed on algae, and culturing the organisms that feed on the algae in the eutrophic water, thereby reducing the algal biomass. The methods of the invention employ organisms of various trophic levels to consume algae in waters rich in nutrients. In another embodiment, the invention provides methods for modifying the diversity of algal species in a body of water, particularly when the aquatic condition is conducive to development of an algal bloom. In yet another embodiment, the invention provides methods for increasing the algal biomass in a body of oligotrophic water. The systems of the invention are based on adapting an offshore oil/gas platform for use in the methods of the invention. The term “hydrocarbon production installation” is used herein to refer generally to any structure that is installed offshore for the drilling of wells or for the production and processing of oil and/or gas, which includes but is not limited to, various types of platforms as well as pipelines that connect the platforms to other facilities onshore and on the seabed.

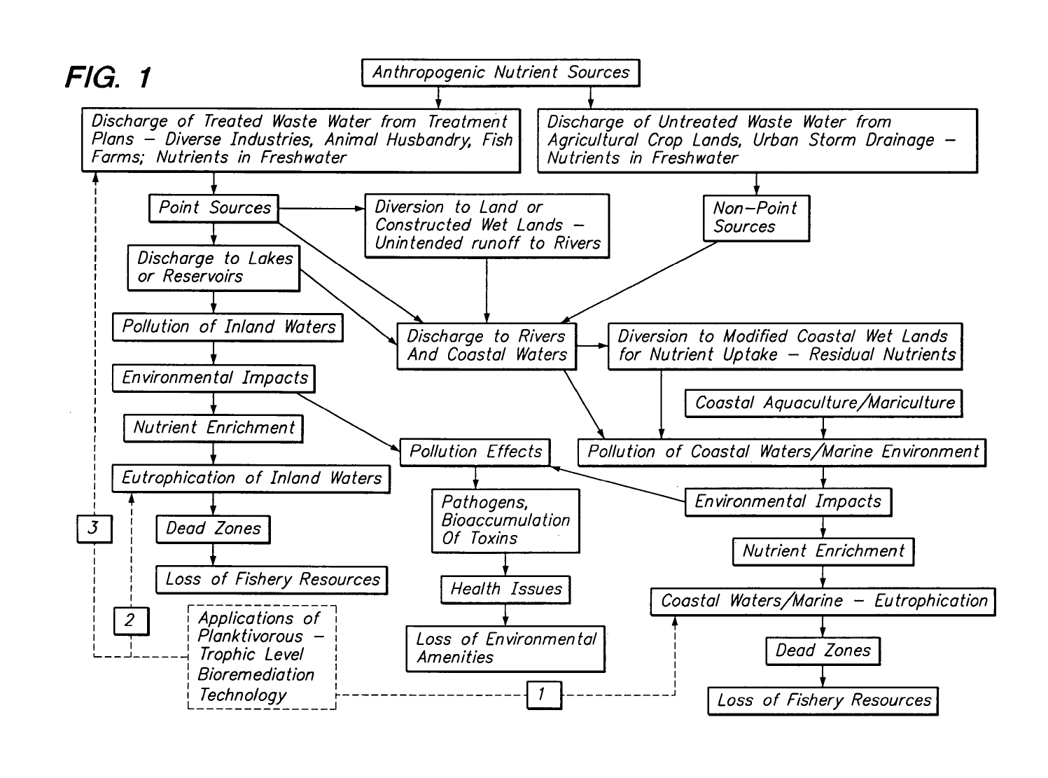

The over-enrichment of water by nutrients, referred to herein as “eutrophication” degrades many aquatic ecosystems worldwide. The current conceptual model of eutrophication recognizes an interacting set of direct and indirect responses of an aquatic ecosystem to nutrient enrichment. Nutrient input stimulates accumulation of phytoplankton biomass, followed by vertical flow of algal-derived organic matter to bottom waters and the sediments, decomposition of the organic matter by bacteria which use up oxygen dissolved in the water. Indirectly, eutrophication affects water transparency, distribution of vascular plants and biomass of macroalgae, nutrient cycling, nutrient ratios, plankton community composition, frequency of toxic/harmful algal blooms, habitat quality for metazoans, and reproduction/growth/survival of pelagic and benthic invertebrates. (2001, Cloem, Our evolving conceptual model of the coastal eutrophication problem, Mar Ecol Prog Ser 210:223-253). FIG. 1 shows some of the events that lead to loss of fishery resources and environmental amenities, starting from anthropogenic activities that produce nutrients, eutrophication, and the development of dead zones. The inventors believe that an understanding of the relationships of various elements in an eutrophic system offers the possibility of intervention with a view towards abating the deleterious effects of eutrophication.

Regulation of algal biomass in a body of water involves removing excess biomass or stimulating algal growth, depending on the nutrient level of the water. The inventors recognizes that the presence of an assemblage of organisms that feed on algae in eutrophic water can be used to counter an increase in algae population by the effect of grazing or foraging. Algae can be removed efficiently by the organisms provided that they are deployed and allowed to feed in eutrophic water where the oxygen level is not limiting for the organisms and the level of algal toxin, if present, does not harm the organisms. Where one or more macronutrients and/or micronutrients are limiting the growth of algae or certain species of algae in a body of water, the systems of the invention are used to deliver the deficient macronutrients and/or micronutrients to the body of water, to stimulate growth or to stabilize the algal population. The regulation of algal biomass in a geographic area is essentially a large scale environmental remediation project. The invention takes advantage of the location and infrastructure of numerous oil and natural gas platforms present in areas proximate to bodies of eutrophic water, where algal blooms, harmful algal blooms and hypoxic zones occur regularly or persistently. The invention is, however, not limited to using oil and gas platforms located in areas that are prone to formation of algal bloom, harmful algal bloom and/or hypoxic zone.

In one embodiment of the invention, the methods use organisms of various trophic levels to consume algae in waters rich in nutrients. Primarily, the organisms are planktivores that occupy the second trophic level, and feed on phytoplankton that are of the first level. When algal blooms occur in natural water bodies, there is a trophic-level association of diverse animal forms, between zooplankton and fishes that consume algae and higher invertebrates, and vertebrates that feed on the zooplankton and smaller species of vertebrates. Within this trophic system, there is a balance in population between the feeding groups that is driven by algal productivity. Nutrient loading causes algal productivity to proceed at rates that are many orders of magnitude higher than consumption by animal forms. Zooplankton blooms generally follow the peak in phytoplankton blooms which begin to diminish due to overgrazing or nutrient depletion. The invention takes into account these relationships in population dynamics of blooms and bloom-induced trophic associations.

The invention is supported by the observation that benthic bivalve filter feeders play the role of a biological, locale-specific attribute which modulates the response of an estuarine-coastal ecosystem to nutrient enrichment, leading to large differences among such ecosystems in their sensitivity to nutrients. For example, one of the preferred predictor of chlorophyll a (chl a) concentration in Danish estuaries, a measure of algal biomass concentration, is the biomass of mussels. Another example shows that the balance between phytoplankton production and loss to benthic feeders can be disrupted by the colonization of an ecosystem by nonindigenous suspension feeders. This occurred in northern San Francisco Bay when the Asian clam Potamocorbula amurensis became widely established in 1987; and since then, chlorophyll biomass has been persistently low and primary production has been reduced 5-fold (Alpine & Cloern, 1992, Trophic interactions and direct physical effects control phytoplankton biomass and production in an estuary. Limnol Oceanogr 37:946-955). These phenomena occur where there is an interaction between the pelagic and benthic zones involving a large number of benthic bivalve suspension feeders removing phytoplankton from the water.

Both fishes and shellfishes can be used in the invention to populate a body of eutropic water. The organisms are cultured in the eutrophic water so that the organisms consume the algae, thereby reducing the algal biomass. By any methods known in the art, the fishes and shellfishes are concentrated in, confined to, contained in, held in, or directed to the water proximate to a hydrocarbon production installation. The culture can be maintained until either the algal biomass is reduced to a desired level, or the organisms have grown to a desirable size and are ready to be harvested. The body of eutrophic water can be restocked with the organisms that are hatched and/or reared on the deck of a platform to begin another round of culture. The steps of introducing, culturing and harvesting the organisms in the eutrophic water can be conducted repeatedly for multiple rounds (e.g., at least one round), or for as long as the need to reduce algal biomass exists. It is also contemplated that specific algal species can be cultured and used to seed the water in order to steer a growing population of algae towards a more desirable distribution of species, for example, a population comprising algal species that compete effectively for nutrients against certain other species that produce toxins and that can cause a harmful algal bloom. Cultures of beneficial algal species can be maintained and produced on the deck of a platform.

The invention also provides that fishes that are fed on algae can be used as food fish, aquaculture feed, and also as biomass for making biofuels or industrial feedstocks. The shellfishes of the invention can be sold for human consumption or rendered. The invention also provides the use of the planktivorous fishes as live feed to piscivorous fishes that can also be cultured in close proximity in separate enclosures. Such piscivorous fish generally have a higher market value than planktivores, and can thereby provide an additional revenue stream to support the operation.

The invention also provides oil and gas platform-based systems for regulating algal biomass. Such systems comprise a plurality of modules for managing nutrients, algae, and fishes/shellfishes in the water proximate to a hydrocarbon production installation. The modules are installed on a hydrocarbon production installation or extensions thereof. It is contemplated that a network of such platform-based systems can remediate the aquatic environment of a wide geographic area, such as the Northern coastal areas of the Gulf of Mexico.

Modules for managing nutrient levels are provided which include a network of pipelines that connect hydrocarbon production installations with one another, with floating production vessels, or with shore facilities; and risers that fluidically connect the seabed to the surface or water at any intermediate depth. Water comprising nutrients and algae can be transported in the network to various locations in a geographic area. The algae modules can comprise apparatus for sampling water, imaging subsystems for monitoring algal growth and species diversity, facilities to grow algae, apparatus for concentrating and distributing algae, and a network of pipes for transporting algae. The systems also comprise an assemblage of fishes and/or shellfishes that are active at multiple trophic levels. When the fishes/shellfishes are introduced into a body of eutrophic water, they are contained in enclosures that are operably associated with a hydrocarbon producing installation. Different species of fishes and shellfishes at various ages or developmental forms can be used in combination or in sequence to remove algae from the eutrophic water. The aquaculture modules of the systems comprise hatchery facilities for hatching and rearing fishes and shellfishes, algal culture facilities, apparatus for feeding the fishes/shellfishes, instruments for monitoring the aquatic environment for fish/shellfish culture, means for recruiting fishes to the enclosure by light, sound, or a chemical gradient, apparatus for aeration of water at various depths, means for transferring fishes/shellfishes, apparatus for harvesting fishes/shellfishes, predator deterrence means, and facilities for processing fish oil and fish meal, and rendering shellfishes. Methods for maintaining stocks of algae, fishes, and/or shellfishes on the deck of a hydrocarbon production installation that is adapted to producing organisms of the appropriate age or developmental stage year round for use in the invention, are also encompassed in the invention.

The culturing of fishes and shellfishes in the body of water can entail replenishing the water in or near the enclosure with algae; and/or maintaining the level of dissolved oxygen in the water in or near the enclosure above hypoxic level (such as about 3 mg per liter). The methods of the invention also comprise transporting a stream of algae-containing and/or oxygenated water from a location distant from the body of water or another water depth by a hydrocarbon production installation, and discharging the stream into or near the enclosure, optionally admixing the stream with water in the enclosure or with water proximate to the enclosure. The admixing serves the purposes of replacing the algae that have been consumed, facilitating gaseous exchange, and diluting algal toxin and waste produced by the organisms. Hydrocarbon production installations such as pipelines, risers, manifolds, pumps, and the likes, can be used to transport the stream of algae-containing and/or oxygenated water. Various techniques for transporting and/or admixing fluids are known in the hydrocarbon production industry and can be applied in the methods of the invention.

The methods of the invention also encompass increasing the algal biomass in a body of water that is oligotrophic (<30×10³ phytoplankton cells per liter). The limited availability of one or more macronutrients and/or micronutrients is usually the cause of low productivity and diminished algal biomass. The primary nutrients responsible for eutrophication are nitrogen and phosphorous, although other micronutrients, such as iron and silicates, are also implicated. Whether primary production by phytoplankton is nitrogen or phosphorous limited is a function of the relative availabilities of the two elements in water. Phytoplankton require approximately 16 moles of nitrogen for every mole of phosphorous they assimilate, i.e., the Redfield ratio of 16:1, (Redfield, 1958, American Scientist, 46:205-222). If the Redfield ratio is less than 16:1, phytoplankton growth will tend to be limited by nitrogen. If the ratio is higher, phytoplankton growth will tend to be phosphorous limited. It has been observed that nitrogen limitation is more prevalent in coastal marine ecosystems than in lakes. The methods of the invention comprise transporting a stream of nutrient-rich or eutrophic water that has a higher concentration of macronutrients and/or micronutrients relative to the body of oligotrophic water, from a distance or a different water depth, and discharging it in the body of oligotrophic water, optionally admixing the stream with the body of oligotrophic water. The increase in nutrients in can also be accomplished by adding the macronutrients (e.g., nitrogen fertilizer and/or phosphorous fertilizer) or micronutrients (e.g., iron and/or silicates) directly to the body of water. Solid or liquid forms of the macronutrients and micronutrients can be used. Non-limiting examples of macronutrients include C, O, H, N, Na, K, Ca, P, S, Mg and Cl. Non-limiting examples of micronutrients include Fe, Zn, Mn, Br, Si, B, Mo, V, Sr, Al, Rb, Li, Cu, Co, I and Se. Optionally, the methods can include adding to or seeding the body of water with, one or more species of algae. The results of addition of nutrients or nutrient-rich water is similar to upwelling that occurs naturally.

In yet another embodiment of the invention, the diversity of algal species in a body of water, such as the water in the vicinity of an oil/gas platform, is modified. Typically, the body of water comprises a growing algal bloom, with more than 10⁵ cells per ml. One of the objectives of the invention is to prevent the dominance within a population of algae by certain algal species that produce toxins in the body of water, e.g., dinoflagellates. The methods comprise altering the concentration of macronutrients and/or micronutrients that are required by certain species of algae in the body of water. The modification can be accomplished by transporting a stream of water or other fluids that has a different concentration of macronutrients and/or micronutrients relative to ambient water, discharging it in or admixing it with a body of water comprising the algae. The modification can also be accomplished by adding the macronutrients or micronutrients directly to ambient water. Optionally, the methods can include adding to or seeding the body of water with, one or more species of algae that are not present in the body of water, or that are not present as a dominant species.

The Mississippi River continental shelf has experienced a change in silicon:dissolved inorganic nitrogen (Si:diN) from 3:1 to 1:1 in the 20^{th} century due to land-use practices in the watershed. Collected data suggests that as the Si:diN ratio drops to 1:1 or below, the food web based on the flow of energy from diatom to zooplankton to higher trophic levels is reduced or disrupted, and the potential for flagellated algal bloom including harmful algal bloom increases (Turner et al., Proc Natl. Acad. Sci. 95:13048-13051, 1998). It has been shown that the supply of iron, relative to the supply of nitrate (NO₃), phosphate (HPO₄), and silicic acid (H₄SiO₄), critically limits the uptake of nitrate by diatoms, especially chain-forming diatoms greater than 8 μm in size, e.g., Chaetoceros, Pseudonitzschia, Coscinodiscaceae, and Navicula species. For example, water in a natural coastal upwelling comprises 15-35 μM. of nitrate, 1.3-2.6 μM of phosphate, 15-45 μM. of silicic acid and concentration of dissolved and particulate iron greater than about 5 nM to greater than about 10 nM. Iron concentration below 1 nM results in unused nitrate and silicic acid and a low abundance of diatoms greater than about 8 μm. (Hutchins et al., Nature 393:561 1998; Bruland et al., Limnol. Oceanogr. 46:1661-1674, 2001). It is contemplated that the systems and methods of the invention can be used to add iron to the water proximate to a platform, such that the water in the photic zone has a iron nanomolar (nM) concentration greater than about 0.1, 0.2, 0.25, 0.5, 0.75, 1, 2, 2.5, 5, 7.5, 10, 15, 20, 25, and 50.

In yet another embodiment of the invention, the systems and methods can be used to remove heavy metal, such as mercury, from contaminated water proximate to a hydrocarbon producing installation. Bacteria and algae take up heavy metal in the water as they grow. The heavy metal accumulates in the fishes that consume the algae. By harvesting fishes that are fed on algae in the contaminated water, the heavy metal is removed from the water and remains in the fishes which can be processed appropriately at low cost.

It is contemplated that the systems and methods of the invention can be used in many of the coastal waters in the United States and worldwide where hydrocarbon production installations are installed. The terms “ocean” and “sea” are used interchangeably, and are taken to represent the bodies of salt water which cover the surface of the earth. The term “coast” and “offshore” are used interchangeably to include all areas between land and ocean, such as but not limited to, estuaries and coastal ocean. A coast can be classified as open continental shelf (e.g., Georgia Bight, Monterey Bay, Louisiana Shelf), coastal embayment (e.g., Massachusetts Bay, Buzzards Bay, Long Island Sound), river plume estuary (e.g., Mississippi River Plume), coastal plain or drowned river valley estuary (e.g., Chesapeake Bay, Hudson River, Charleston Harbor, Choctawhatchee Bay, Perdido Bay, Apalachee Bay), coastal plain salt marsh estuary (e.g., Plum Island Sound, North Inlet, Duplin River, Pensacola Bay), lagoon (e.g., Padre Island, Pamlico Sound, Apalachicola Bay), fjord estuary (e.g., Penobscot Bay), coral reef system (e.g., Kaneohe Bay), tectonically-caused estuary (e.g., San Francisco Bay, Tomales Bay), large river with non-drowned river estuary (e.g., Columbia River), seagrass-dominated estuary (e.g., Tampa Bay, lower Perdido Bay), rocky-intertidal macroalgae dominated estuary (e.g., Casco Bay). Exemplary areas where algal blooms and hypoxic zone appear regularly include but are not limited to, the Gulf of Mexico, Kattegat near Sweden and Denmark, Baltic Sea, Bohai Sea, Taihu (or Lake Tai), and Black Sea. The inner to mid-continental shelf (depths of 5 to 60 m) of the Northern Gulf of Mexico, from the Mississippi River delta westward to the upper Texas coast, is the site of the largest hypoxic bottom water in the western Atlantic Ocean coastal zone (Rabalais et al., 2001, J Environ Qual 30:320-329).

Technical and scientific terms used herein have the meanings commonly understood by one of ordinary skill in the art to which the present invention pertains, unless otherwise defined. Reference is made herein to various methodologies known to those of skill in the art. Publications and other materials setting forth such known methodologies to which reference is made are incorporated herein by reference in their entireties as though set forth in full. The practice of the invention will employ, unless otherwise indicated, techniques of chemical engineering, biology, and the aquaculture industry, which are within the skill of the art. Such techniques are explained fully in the literature, e.g., Aquaculture Engineering, Odd-Ivar Lekang, 2007, Blackwell Publishing Ltd.; Handbook of Microalgal Culture, edited by Amos Richmond, 2004, Blackwell Science; Cage Aquaculture. M. Beveridge, third edition, 2004, Blackwell Publishing Ltd., each of which are incorporated by reference in their entireties.

As used herein, “a” or “an” means at least one, unless clearly indicated otherwise. The term “about,” as used herein, unless otherwise indicated, refers to a value that is no more than 20% above or below the value being modified by the term. As it is well known to those skilled in the art, a variety of geographic and oceanographic conditions precludes an absolute quantitative measurement of the terms “deep,” “fresh,” “warm” or “cold.” Therefore, these terms are used in the comparative sense. The term “ambient” is used to describe the surrounding water at a given location or depth. The term “proximate to” is used herein to describe, in context, a functionally effective distance relative to a location, such as a platform of the invention, and the term relates to the dimensions of a body of water being addressed in context, such as the circumference and the size of an area about a location. Without limitation, the term “proximate to” means a distance of about 1 to 10 meters, about 15, 20, 25, 50, 75, 100, 200, 250, 500, 750, 1000, 1500, 2000, 2500, 3000, or 5000 meters, or any intermediate distances.

For clarity of disclosure, and not by way of limitation, the detailed description of the invention is divided into the subsections which follow.

### 5.1. Ecological Effects of Eutrophication

The terms “eutrophic” and “eutrophication” are used herein to describe the presence of an abundance of nutrients in a body of water, i.e., nutrient enrichment. For many aquatic ecosystems, primary productivity is limited by nutrient availability. Therefore, a direct response to nutrient input in these ecosystems is an increase in phytoplankton biomass, leading to the formation of an algal bloom. A body of water with productivity greater than about 300 g C m⁻² year⁻¹ and exhibits at least one of the following characteristics is referred to as a body of eutrophic water, that forms an eutrophic zone in a body of water. Some of the characteristics of eutrophic water, relative to non-eutrophic water, include but are not limited to, increased phytoplankton productivity, high chlorophyll a concentration, decreased light availability to benthic zone, high epiphytic growth rate, high non-perennial macroalgae growth rate, changes in dominance from benthic algae to pelagic algae, and a shift in dominance from diatoms to dinoflagellates. The invention can be used to reverse at least some of the changes observed in the aquatic environment that results from eutrophication.

Many coastal waters are shallow enough that benthic plant communities thrive where sufficient light penetrates the water column to the seafloor. Benthic vascular plants (seagrasses) and perennial macroalgae are more adapted to low nutrient environments than phytoplaktons and ephemeral macroalgae. An increase in nutrient input results in the progressive selection for fast-growing algae that are best adapted to high-nutrient conditions, at the expense of slower-growing seagrasses and perennial macroalgae. Phytoplankton biomass can reduce light penetration and epiphytic microalgae becomes more abundant on seagrass leaves in eutrophic water contributing to light attenuation. Ephemeral macroalgae, such as Ulva, Caldophora, and Chaetomorpha, can form extensive thick mats over the seagrass leading to its disappearance from the seafloor. Loss of benthic seagrasses and macroalgae will result in changes in the associated fauna, and increases sediment resuspension that causes influx of nutrients from the sediment further promoting algal blooms. The accumulation of ephemeral macroalgae is a nuisance to recreational users of beaches and waterways. The invention can be used to prevent or reduce the loss of seagrasses and perennial macroalgae by reducing the phytoplankton biomass.

Plankton species have a wide range of nutrient requirements and the plankton community composition of a coastal region can be directly changed by eutrophication. A limitation of silicates in the water restricts the growth of diatoms and/or the amount of silicon in their bodies. It has been observed that as the size of the diatom population in a water column falls, an increase in the number of flagellates is detected. Among the many species of algae that thrives in a body of water, some can produce toxins that are harmful to other organisms that either ingest these species or share the same aquatic environment. For example, the species Chaetoceros has been associated with the deaths of fanned salmon, they have long, barbed spines that lodge in fish gills, causing a buildup of mucus, degeneration of the respiratory system, and eventual death by suffocation. Some species interfere with fish reproduction. When one or more species of algae that can produce toxins or cause harm to other marine life and humans, proliferate and become numerically dominant in an eutrophic zone, a harmful algal bloom (HAB) is formed. The invention can be used to prevent the formation of a harmful algal bloom or to reduce the algal biomass in a harmful algal bloom.

Eutrophication is also accompanied by an increased demand for oxygen, due in part to respiration of the increased biomass of plants and animals in the nutrient-loaded system, but mostly to respiration of bacteria in the water column and sediments that consume the organic matter produced by or resulting from death of, the plant and animal biomass. If the loss of oxygen is not offset by the introduction of additional oxygen by photosynthesis or mixing, then hypoxia or anoxia occurs. Dissolved oxygen less than about 2.0 to 3.0 mg per liter is referred to herein as hypoxia. Anoxia is a form of hypoxia when biologically useable dissolved oxygen is completely absent. Hypoxia and anoxia are more likely to occur in warmer months because of thermal stratification of the water body that prevents mixing of oxygen-rich surface water with bottom water. The occurrence of areas of hypoxia near the coasts can kill marine life, disrupt their migration and habitat, and change the benthic community structure. The invention can be used to prevent hypoxia in an eutrophic zone, which includes removing algal biomass in the body of water that lies above the hypoxic zone

### 5.2 Algal Bloom

As used herein the term “algae” refers to any organisms with chlorophyll and a thallus not differentiated into roots, stems and leaves, and encompasses prokaryotic and eukaryotic organisms that are photoautotrophic or photoauxotrophic. The terms “microalgae” and “phytoplankton” refer to any microscopic algae, photoautotrophic or photoauxotrophic protozoa, and cyanobacteria (formerly classified as Cyanophyceae). These microscopic aquatic organisms are also referred to, with other microscopic organisms, as plankton.

The composition and numbers of phytoplankton communities depend on a balance of local factors. Phytoplankton inhabit all types of aquatic environment, including but not limited to freshwater (less than about 0.5 parts per thousand (ppt) salts), brackish (about 0.5 to about 31 ppt salts), marine (about 31 to about 38 ppt salts), and briny (greater than about 38 ppt salts) environment. Field observations indicate the distribution of sizes of a phytoplankton community often varies with resource availability and hydrographic conditions. It has been observed generally that small phytoplankton are dominant under oligotrophic conditions, and large phytoplankton are more abundant in eutrophic water. The methods of the invention can be used to remove plankton, phytoplankton and/or zooplankton, of various sizes, ranging in dimensions from about 200 to 2000 μm, about 20 to 200 μm, about 2 to 20 μm, or about 0.2 to 2 μm.

Algal species can be identified by microscopy and enumerated by counting or flow cytometry, which are techniques well known in the art. Chlorophyll a is a commonly used indicator of algal biomass. However, it is subjected to variability of cellular chlorophyll content (0.1 to 9.7% of fresh algal weight) depending on algal species. The estimated biomass value can be calibrated based on the chlorophyll content of the dominant species within a population. Published correlation of chlorophyll a concentration and biomass value can be used in the invention. Chlorophyll a concentration is to be measured within the euphotic zone. The euphotic zone is the depth at which the light intensity of the photosynthetically active spectrum (400-700 nm) equals 1% of the subsurface light intensity.

Nutrient over-enrichment destabilizes plankton populations by allowing certain species to compete successfully against all other species present. Apparently, initiation of an algal bloom (including a harmful algal bloom) is dependent on a series of events or a set of optimal conditions. Thus, a bloom can be circumvented by disrupting at least one event in the sequence, or altering the optimal conditions. A body of eutrophic water comprising more than about 1×10⁵ algal cells per ml can develop a bloom under certain conditions. For example, the body of eutrophic water can comprise more than about 2×10⁵, about 5×10⁵, about 8×10⁵ algal cells per ml. The term “algal bloom” as used herein refers to the presence of algae in a body of water comprising at least about 1×10⁶ algal cells per ml or greater, such as but not limited to about 5×10⁶ algal cells per ml, about 10⁷ algal cells per ml, about 5×10⁷ algal cells per ml, and about 10⁸ algal cells per ml.

An algal bloom comprises one or several numerically dominant species of algae. Dominant species in an algal bloom is referred to as a bloom species. A dominant species is one that proliferates and thus ranks high in the number of algal cells, e.g., the top one to five species with the highest number of cells relative to other species. The one or several dominant algae species may constitute greater than about 10%, about 20%, about 30%, about 40%, about 50%, about 60%, about 70%, about 80%, about 90%, about 95%, about 97%, about 98% of the algae present in the culture. In certain embodiments, several dominant algae species may each independently constitute greater than about 10%, about 20%, about 30%, about 40%, about 50%, about 60%, about 70%, about 80% or about 90% of the algae present in the culture. Many other minor species of algae may also be present in such culture but they may constitute in aggregate less than about 50%, about 40%, about 30%, about 20%, about 10%, or about 5% of the algae present. In various embodiments, one, two, three, four, or five dominant species of algae are present in a body of eutrophic water or in a bloom. Accordingly, a body of eutrophic water comprising algae or an algae bloom can be described and distinguished from another body of water or bloom by the dominant species of algae present. The population of algae present in a body of eutrophic water can be described by the percentages of cells that are of dominant species relative to minor species, or the percentages of each of the dominant species.

In the context of coastal systems, of the estimated total number of marine phytoplankton (about 5000), some 300 species are known to occur at numbers high enough to discolor water (Sournia et al., 1991, J Plankton Res 13; 1093-1099). About 40 to 50 of these species produce toxins that can affect marine plants and animals (Hallegraeff 1995, Manual on harmful Marine Microalgae. IOC manual and guides 33. UNESCO, Paris). The algal species that discolor water or produce harmful effects described in the above references can be removed from water by the methods of the invention. Specific examples of such algal species are described below.

In various embodiments, one or more species of algae belonging to the following phyla can be removed by the methods of the invention: Cyanobacteria, Cyanophyta, Prochlorophyta, Rhodophyta, Glaucophyta, Chlorophyta, Dinophyta, Cryptophyta, Chrysophyta, Prymnesiophyta (Haptophyta), Bacillariophyta, Xanthophyta, Eustigmatophyta, Rhaphidophyta, and Phaeophyta. The methods of the invention are most effective at removing algae that are microscopic, including algae in unicellular and colonial forms.

In certain embodiments, the algae in eutrophic water comprises cyanobacteria (also known as blue-green algae) from one or more of the following taxonomic groups: Chroococcales, Nostocales, Oscillatoriales, Pseudanabaenales, Synechococcales, and Synechococcophycideae. Non-limiting examples include Gleocapsa, Pseudoanabaena, Oscillatoria, Microcystis, Synechococcus and Arthrospira species.

In certain embodiments, the algae in eutrophic water comprise algae from one or more of the following taxonomic classes: Euglenophyceae, Dinophyceae, and Ebriophyceae. Non-limiting examples include Euglena species and the freshwater or marine dinoflagellates.

In certain embodiments, the algae in eutrophic water comprise freshwater, brackish, or marine diatoms from one or more of the following taxonomic classes: Bacillariophyceae, Coscinodiscophyceae, and Fragilariophyceae. Preferably, the diatoms are photoautotrophic or auxotrophic. Non-limiting examples include Achnanthes (e.g., A. orientalis), Amphora (e.g., A. coffeiformis strains, A. delicatissima), Amphiprora (e.g., A. hyaline), Amphipleura, Chaetoceros (e.g., C. wighami, C. subtilis, C. affinis, C. muelleri, C. gracilis), Caloneis, Camphylodiscus, Cyclotella (e.g., C. cryptica, C. meneghiniana), Cricosphaera, Cymbella, Diploneis, Entomoneis, Fragilaria, Hantschia, Gyrosigma, Melosira, Navicula (e.g., N. acceptata, N. biskanterae, N. pseudotenelloides, N. saprophila), Nitzschia (e.g., N. dissipate, N. communis, N. inconspicua, N. pusilla strains, N. microcephala, N. intermedia, N. hantzschiana, N. alexandrina, N. quadrangula), Phaeodactylum (e.g., P. tricornutum), Pleurosigma, Pleurochrysis (e.g., P. carterae, P. dentata), Selenastrum, Surirella and Thalassiosira (e.g., T. weissflogii).

The following algae are bloom species that are found in algal bloom, including harmful algal bloom: cyanobacteria (e.g., Anabaena species, Aphanizomenon species, Microcystis species (e.g., M. aeruginosa), Merismopedia tenuissima); dinoflagellates (e.g., Noctiluca scintillans (responsible for red tides in many parts of the world), Alexandrium species (e.g., A. fundyense, A. tamarense, A. monilatum), Prorocentrum species (e.g., P. micans, P. lima, P. minimum, P. triestinum), Gymnodinium species (e.g., G. breve (also known as Karenia brevis commonly found in Florida red tide), G. catenatum, G. mikimotoi), Ceratium species (e.g. C. furca, C. hircus), Karlodinium veneficum, Pfiesteria species (e.g., P. shumwayae, P. piscicida), Amphidinium operculatum, Cochlodinium heterolobatum, Dinophysis sp. (e.g., D. acuminata, D. acuta, D. caudata, D. fortii, D. norvegica), Gyrodinium aureolum, Scrippsiella trochoidea, Akashiwo sanguinea); Raphidophyte (e.g., Heterosigma akashiwo, Chattonella species (e.g., C. verruculosa, C. antique, C. marina), Fibrocapsa japonica); diatoms (e.g., Skeletonema species (e.g., S. costatum, S. potamus), Synedropsis species, Pseudonitzschia species (e.g., P. pseudodelicatissima, P. seriata, P. pungens), Leptocylindrus danicus, Chaetoceros species (e.g., C. curvisetus, C. pseudocurvisetus), Biddulphia sinensis, Eucompia zoodiacus); Haptophytes (e.g, Dictyocha fibula, Phaeocystis species, (e.g., P. globosa, P. pouchetii)); brown tide species (Nannochloris atomus, Stichococcus species, Aureococcus anophagefferens) and Protoperidinium brevipes. Additional exemplary species found in algal blooms in the Gulf of Mexico include but are not limited to Miraltia throndsenii, Guinardia delicatula, Spermatozopsis exsultans, Urosolenia eriensis, Nitschia reversa, Cyclotella chocotawhatcheeana, Cryptomonas species, Pedinophora species.

In one embodiment of the invention, satellite or aerial remote sensing measurement of ocean color provides data that are complementary to in-situ measurement of water samples for monitoring algal bloom. Plankton-imaging instruments that allow counting and visual analysis of plankton in water samples can be deployed on or near hydrocarbon production installation. The instruments can be installed on a structural member of a platform, enclosures that are associated with a platform, and/or buoys within serviceable distance from the hydrocarbon production installation. Many such imaging systems have been described in the art and can be adapted for use as a part of a system of the invention. See, for example, Davis et al., 2004. Real-time observation of taxaspecific plankton distributions: An optical sampling method. Marine Ecology Progress Series 284:77-96; Benfield et al., 2007. RAPID: Research on Automated Plankton Identification, Oceanography, 20:172-187. Powerful remote sensing technologies are available to facilitate detection of algal bloom and identification of eutrophic waters that may develop a bloom. For example, at least four satellites have been commonly used for chlorophyll and phytoplankton mapping: AVHRR, SEAWIFS, LANDSAT and MODIS. Imaging spectrometers (also known as hyperspectral sensors) used in remote sensing simultaneously collect spectral data as both images and as individual spectra. A range of techniques, including fourth-derivative analysis, have been put into operational practice for analysis of algal blooms. For an overview of this technology, see, for example, Remote sensing of algal bloom dynamics by Richardson, LL in Bioscience Vol. 46, no. 7, pp. 492-501. July-August 1996; and US patent publication US 2005/0164333 A1

Radiation leaving a water body is a function of reflection, absorption, and transmission of the optically active constituents in a water body. The reflectance properties of a body of water are typically determined by concentrations of algae and photosynthetic bacteria containing a variety of photosynthetic and photoprotective pigments, such as but not limited to, chlorophyll a, b, and c, generally, gyroxanthin in Karenia brevis, fucoxanthin in Phaeophyceae, and peridinin in dinoflagellates). Chlorophyll a is the primary pigment in phytoplankton that absorbs light for use in photosynthesis. Chlorophyll a absorption peaks in the visible light between 400 and 475 nm and around 665 nm; it reflects most of the light with wavelengths between 475-550 nm. Chlorophyll c, a major secondary pigment, peaks in absorption at 475 and 650 nm, but reflects light from about 500-550 nm. Cyanobacteria is characterized by the presence of cyanophycoerythrin and/or cyanophycocyanin with absorption maxima in the range of 620 to 630 nm. As the individual phytoplankton pigments are characterized by their unique light absorption features, this property allows detection and identification of algal blooms by ocean color spectra. The reflectance spectrum of a body of water consists of overlapping spectral features caused by the presence of individual pigments related to single species and mixtures of species. It has been shown that certain characteristic peaks within the individual pigments can be deconvolved from reflectance spectra, thus allowing mapping of the spatial distribution of specific pigments and give information about the abundance and taxonomic composition of algae. It is contemplated that the use of regional reflectance data including analysis of hyperspectral data be incorporated into methods of the invention to assist in locating and identifying algal bloom. Any of numerous algorithms for converting spectral data to algal biomass data can be used. The light measurement device can be a photosensor, camera, digital camera and video camera, etc. The measurement device may be placed in any position from which it can sense the required light wavelengths, such as on a satellite, an aircraft, a buoy, a boat, a light house, or a handheld device. Any commercially available equipment for collecting and analyzing wavelength and geographic data can be used in the present invention.

In connection with this aspect of the invention, the systems of the invention comprise data systems which include but are not limited to a plankton-imaging instrument, a data processing device having programming instructions for counting plankton, optionally classifying the plankton taxonomically, and applying an algorithm that convert image data into algal biomass data; a transmitter for transmitting data from the imaging device to a site remote from the site where the water is sampled and/or where imaging takes place; and a report generator for producing a report of the amounts of algal biomass in the water at a locale. In addition, the systems comprise a light measurement device adapted to sense and record and/or transmit the light wavelength reflected from water; a data processing device having programming instructions for applying an algorithm that convert image and/or spectral data into algal biomass data, optionally linked to geographic data; a transmitter for transmitting data from the measuring device or processor to a site remote from the site where the measurement takes place; and a report generator for producing a report of the amounts of algal biomass in the water over an area. Optionally, the data systems of the invention comprise a means for integrating algal biomass data obtained from imaging water samples, from satellite or aerial remote sensing, and historical data, and present them on a map for visual inspection and reporting. The report generator may be any device that is adapted to place the data into a tangible medium, such as a printer, disc burner, flash memory, magnetic storage media, etc.

### 5.3 Organisms

The organisms useful in the invention include fishes and shellfishes. As used herein, the term fish refers to a member or a group of the following classes: Actinopteryii (i.e., ray-finned fish) which includes the division Teleosteri (also known as the teleosts), Chondrichytes (e.g., cartilaginous fish), Myxini (e.g., hagfish), Cephalospidomorphi (e.g., lampreys), and Sarcopteryii (e.g., coelacanths). The teleosts comprise at least 38 orders, 426 families, and 4064 genera. Some teleost families are large, such as Cyprimidae, Gobiidae, Cichlidae, Characidae, Loricariidae, Balitoridae, Serranidae, Labridae, and Scorpaenidae. In many embodiments, the invention involves bony fishes, such as the teleosts, and/or cartilaginous fishes. When referring to a plurality of organisms, the term “fish” is used interchangeably with the term “fishes” regardless of whether one or more than one species are present, unless clearly indicated otherwise. As used herein, the terms “shellfish” and “shellfishes” refers to various species of molluscs, crustaceans, and echinoderms that feed on plankton. The term “shellfish” is used herein as singular and plural interchangeably with the term “shellfishes” regardless of whether one or more than one species are present, unless clearly indicated otherwise.

In an aquatic environment, fish occupy various trophic levels, such as carnivores (e.g., piscivores), herbivores, planktivores, detritivores, and omnivores. Fishes that can be used in the present invention feed on algae, but it is not required that they feed exclusively on algae. Planktivores are preferably used. They can be planktivores that feed on both phytoplankton and zooplankton. Omnivores and herbivores can also be used to remove the algae. Many of the planktivores and omnivores are filter feeders. They can be pelagic filter feeders or benthic filter feeders. Many species of planktivores develop specialized anatomical structures to enable filter feeding, e.g., gill rakers and gill lamellae. Generally, the sizes of such structures relative to the dimensions of the plankton in the water affect the diet of a planktivore. Fish having more closely spaced gill rakers with specialized secondary structures to form a sieve are typically phytoplanktivores. Others having widely spaced gill rakers with secondary barbs are generally zooplanktivores. In the case of piscivores, the gill rakers are generally reduced to barbs. Gill lamellae are structures found in shellfishes adapted for filter feeding.

Gut content analysis can determine the diet of an organism used in the invention. Techniques for analysis of gut content of fish and shellfish are known in the art. As used herein, a planktivore is a phytoplanktivore if a population of the planktivore, reared in eutrophic water with non-limiting quantities of phytoplankton and zooplankton, has on average more phytoplankton than zooplankton in the gut. Under similar conditions, a planktivore is a zooplantivore if the population of the planktivore has on average more zooplankton than phytoplankton in the gut. Gut content analysis can also reveal the dimensions of the plankton ingested by the planktivore and the preference of the planktivore for certain species of algae. Knowing the average dimensions of ingested plankton, the preference and efficiency of the planktivore for a certain size class of plankton can be determined. The size preference of a planktivore can be used to match the dimensions of algae in the eutrophic water. The species preference can also be used to match the dominant species of algae in the eutrophic water. Such information on the diet of a planktivore can be useful in choosing the plurality of planktivores to be deployed given the characteristics of the algae in a body of eutrophic water. By matching the preferred size range of algae and/or the species preference of a planktivore with the algae population in the water, the efficiency of the methods of the invention can be improved.

Many plankton feeders go through ontogenetic changes that include their feeding habits. The diet changes are associated with ontogenetic developments in eyesight, locomotion, mouth dimensions, dentition, and gut dimension. As feeding habits change when an organism hatches and grows from a larva into a juvenile, and then into an adult, it can change from one type of feeder to another, e.g., from a zooplanktivore to a phytoplanktivore, or from a phytoplanktivore to a zooplanktivore. It is contemplated that the efficiency of the methods of the invention can be improved by using planktivores of an age or ontogenetic form that matches the characteristics of the algae in the eutrophic water. For example, where the dimensions of a dominant species in the eutrophic water or the bloom species in an algal bloom, a harmful algal bloom or a hypoxic zone fall in one of the following ranges: from about 200 to 2000 μm, about 20 to 200 μm, about 2 to 20 μm, or about 0.2 to 2 μm, it would be advantageous to use planktivores that ingest efficiently or prefer plankton with similar dimensions. In certain embodiments of the invention, the plurality of organisms used in the invention are planktivores, including but not limited to obligate planktivores. In addition to planktivores, omnivores, herbivores and/or detritivores can also be used in the methods of the invention. In certain embodiments, one or several major species of the planktivores are phytoplanktivores. In certain embodiments, zooplanktivores are less preferred. In certain embodiments, one or several species of the planktivores are zooplanktivores. In certain embodiments, piscivores are used to harvest other fishes in the systems of the invention. In certain embodiments, detritivores are used to remove solid waste produced by other fishes in the systems. In certain embodiments, carnivores or piscivores are less preferred.

Fishes useful for the invention can be obtained from fish hatcheries or collected from the wild. The fishes may be fish fry, juveniles, fingerlings, or adult/mature fish. By “fry” it is meant a recently hatched fish that has fully absorbed its yolk sac, while by “juvenile” or “fingerling” it is meant a fish that has not recently hatched but is not yet an adult. In certain embodiments of the invention, fry are used. In certain embodiments of the invention, juveniles that have metamorphosed are used. Any fish aquaculture techniques known in the art can be used to stock, maintain, reproduce, and gather the fishes used in the invention. Depending on the local environment and the type of fish used, the fish can be introduced at various density from about 50 to 100, about 100 to 300, about 300 to 600, about 600 to 900, about 900 to 1200, and about 1200 to 1500 individuals per m².

One or more species of fish can be used to remove the algae in eutrophic water. In one embodiment of the invention, the population of fish comprises wild fishes, farmed fishes, and/or genetically improved fish. In another embodiment, the fish population is mixed and thus comprises one or several major species of fish including wild fish, farmed fish, and/or genetically improved fish. A major species is one that ranks high in the head count, e.g., the top one to five species with the highest head count relative to other species. In a preferred embodiment, at least one breed of genetically improved fish, considered a species in this context, is a major species in the population. The one or several major fish species may constitute greater than about 10%, about 20%, about 30%, about 40%, about 50%, about 60%, about 70%, about 75%, about 80%, about 90%, about 95%, about 97%, about 98% of the fish present in the population. In certain embodiments, several major fish species may each constitute greater than about 10%, about 20%, about 30%, about 40%, about 50%, about 60%, about 70%, or about 80% of the fish present in the population. In various embodiments, one, two, three, four, five major species of fish are present in a population. Accordingly, a mixed fish population or culture can be described and distinguished from other populations or cultures by the major species of fish present. The fish population or culture can be further described by the percentages of the major and minor species or the breed(s) of genetically improved fishes, or the percentages of each of the major species. It is to be understood that mixed cultures having the same genus or species may be different by virtue of the relative abundance of the various genus and/or species present.

Fish inhabit most types of aquatic environment, including but not limited to freshwater, brackish, marine, and briny environments. As the present invention is practiced in many of such aquatic environments, any stenohaline species, euryhaline species, marine species, species that grow in brine, and/or species that thrive in varying and/or intermediate salinities, can be used. Fishes from tropical, subtropical, temperate, polar, and/or other climatic regions can be used. Fishes that live within the following temperature ranges can be used: below 10° C., 9° C. to 18° C., 15° C. to 25° C., 20° C. to 32° C. In one embodiment, fishes indigenous to the region at which the methods of the invention are practiced, are used. Preferably, fishes from the same climatic region, same salinity environment, or same ecosystem, as the algae are used. Most preferably, the algae and the fishes are derived from a naturally occurring trophic system.

Fishes from different taxonomic groups can be used in the methods of the invention. It should be understood that, in various embodiments, fishes within a taxonomic group, such as a family or a genus, can be used interchangeably in various methods of the invention. The invention is described below using common names of fish groups and fishes, as well as the scientific names of exemplary species. Databases, such as FishBase by Froese, R. and D. Pauly (Ed.), World Wide Web electronic publication, www.fishbase.org, version (06/2008), provide additional useful fish species within each of the taxonomic groups that are useful in the invention. It is contemplated that one of ordinary skill in the art could, consistent with the scope of the present invention, use the databases to specify other species within each of the described taxonomic groups for use in the methods of the invention.

In certain embodiments of the invention, the fishes used in the invention are in the order Acipeneriformes, such as but not limited to, sturgeons (trophic level 3) e.g., Acipenser species, Huso huso, and paddlefishes (plankton-feeder), e.g., Psephurus gladius, Polyodon spathula, and Pseudamia zonata.

In certain embodiments of the invention, the fishes used in the invention are in the order Clupiformes which include the following families: Chirocentridae, Clupeidae (menhadens, shads, herrings, sardines, hilsa), Denticipitidae, and Engraulidae (anchovies). Exemplary members within the order Clupiformes, i.e., the clupeids, include but are not limited to, the menhadens (Brevoortia species), e.g, Ethmidium maculatum, Brevoortia aurea, Brevoortia gunteri, Brevoortia smithi, Brevoortia pectinata, Gulf menhaden (Brevoortia patronus), and Atlantic menhaden (Brevoortia tyrannus); the shads, e.g., Alosa alosa, Alosa alabamae, Alosa fallax, Alosa mediocris, Alosa sapidissima, Alosa pseudoharengus, Alosa chrysochloris, Dorosoma petenense; the herrings, e.g., Etrumeus teres, Harengula thrissina, Pacific herring (Clupea pallasii pallasii), Alosa aestivalis, Ilisha africana, Ilisha elongata, Ilisha megaloptera, Ilisha melastoma, Ilisha pristigastroides, Pellona ditchela, Opisthopterus tardoore, Nematalosa come, Alosa aestivalis, Alosa chrysochloris, freshwater herring (Alosa pseudoharengus), Arripis georgianus, Alosa chrysochloris, Opisthonema libertate, Opisthonema oglinum, Atlantic herring (Clupea harengus), Baltic herring (Clupea harengus membras); the sardines, e.g., Ilisha species, Sardinella species, Amblygaster species, Opisthopterus equatorialis, Sardinella aurita, Pacific sardine (Sardinops sagax), Harengula clupeola, Harengula humeralis, Harengula thrissina, Harengula jaguana, Sardinella albella, Sardinella janeiro, Sardinella fimbriata, oil sardine (Sardinella longiceps), and European pilchard (Sardina pilchardus); the hilsas, e.g., Tenuolosa species, and the anchovies, e.g., Anchoa species (A. hepsetus, A. mitchilli), Engraulis species, Thryssa species, anchoveta (Engraulis ringens), European anchovy (Engraulis encrasicolus), Australian anchovy (Engraulis australis), Engraulis eurystole, Setipinna phasa, and Coilia dussumieri.

In certain embodiments of the invention, the fishes used in the invention are in the superorder Ostariophysi which include the order Gonorynchiformes, order Siluriformes, and order Cypriniformes. Non-limiting examples of fishes in this group include milkfishes, catfishes, barbs, carps, danios, zebrafish, goldfishes, loaches, shiners, minnows, and rasboras. Milkfishes, such as Chanos chanos, are plankton feeders. The catfishes, such as channel catfish (Ictalurus punctatus), blue catfish (Ictalurus furcatus), catfish hybrid (Clarias macrocephalus), Ictalurus pricei, Pylodictis olivaris, Brachyplatystoma vaillantii, Pinirampus pirinampu, Pseudoplatystoma tigrinum, Zungaro zungaro, Platynematichthys notatus, Ameiurus catus, Ameiurus melas are detritivores. Carps are freshwater herbivores, plankton and detritus feeders, e.g., common carp (Cyprinus carpio), Chinese carp (Cirrhinus chinensis), black carp (Mylopharyngodon piceus), silver carp (Hypophthalmichthys molitrix), bighead carp (Aristichthys nobilis) and grass carp (Ctenopharyngodon idella). Other useful herbivores, plankton and detritus feeders are members of the Labeo genus, such as but not limited to, Labeo angra, Labeo ariza, Labeo bata, Labeo boga, Labeo boggut, Labeo porcellus, Labeo kawrus, Labeo potail, Labeo calbasu, Labeo gonius, Labeo pangusia, and Labeo caeruleus.

In a preferred embodiment, the fishes used in the invention are shiners. A variety of shiners that inhabit the Gulf of Mexico, particularly Northern Gulf of Mexico, can be used. Examples of shiners include but are not limited to, members of Luxilus, Cyprinella and Notropis genus, Alabama shiner (Cyprinella callistia), Altamaha shiner (Cyprinella xaenura), Ameca shiner (Notropis amecae), Ameca shiner (Notropis amecae), Apalachee shiner (Pteronotropis grandipinnis), Arkansas River shiner (Notropis girardi), Aztec shiner (Aztecula sallaei old), Balsas shiner (Hybopsis boucardi), Bandfin shiner (Luxilus zonistius), Bannerfin shiner (Cyprinella leedsi), Beautiful shiner (Cyprinella formosa), Bedrock shiner (Notropis rupestris), Bigeye shiner (Notropis boops), Bigmouth shiner (Hybopsis dorsalis), Blackchin shiner (Notropis heterodon), Blackmouth Shiner (Notropis melanostomus), Blacknose shiner (Can Quebec Notropis heterolepis), Blacknose shiner (Notropis heterolepis), Blackspot shiner (Notropis atrocaudalis), Blacktail shiner (Cyprinella venusta), Blacktip shiner (Lythrurus atrapiculus), Bleeding shiner (Luxilus zonatus), Blue Shiner (Cyprinella caerulea), Bluehead Shiner (Pteronotropis hubbsi), Bluenose Shiner (Pteronotropis welaka), Bluestripe Shiner (Cyprinella callitaenia), Bluntface shiner (Cyprinella camura), Bluntnose shiner (Notropis simus), Bluntnosed shiner (Selene setapinnis), Bridle shiner (Notropis bifrenatus), Broadstripe shiner (Notropis euryzonus), Burrhead shiner (Notropis aspertfrons), Cahaba Shiner (Notropis cahabae), Cape Fear Shiner (Notropis mekistocholas), Cardinal shiner (Luxilus cardinalis), Carmine shiner (Notropis percobromus), Channel shiner (Notropis wickliffi), Chemyfin shiner (Lythrurus roseipinnis), Chihuahua shiner (Notropis chihuahua), Chub shiner (Notropis potteri), Coastal shiner (Notropis petersoni), Colorless Shiner (Notropis perpallidus), Comely shiner (Notropis amoenus), Common emerald shiner (Notropis atherinoides), Common shiner (Luxilus cornutus), Conchos shiner (Cyprinella panarcys), Coosa shiner (Notropis xaenocephalus), Crescent shiner (Luxilus cerasinus), Cuatro Cienegas shiner (Cyprinella xanthicara), Durango shiner (Notropis aulidion), Dusky shiner (Notropis cummingsae), Duskystripe shiner (Luxilus pilsbryi), Edwards Plateau shiner (Cyprinella lepida), Emerald shiner (Notropis atherinoides), Fieryblack shiner (Cyprinella pyrrhomelas), Flagfin shiner (Notropis signipinnis), Fluvial shiner (Notropis edwardraneyi), Ghost shiner (Notropis buchanani), Gibbous shiner (Cyprinella garmani), Golden shiner (Notemigonus crysoleucas), Golden shiner minnow (Notemigonus crysoleucas), Greenfin shiner (Cyprinella chloristia), Greenhead shiner (Notropis chlorocephalus), Highfin shiner (Notropis altipinnis), Highland shiner (Notropis micropteryx), Highscale shiner (Notropis hypsilepis), Ironcolor shiner (Notropis chalybaeus), Kiamichi shiner (Notropis ortenburgeri), Lake emerald shiner (Notropis atherinoides), Lake shiner (Notropis atherinoides), Largemouth shiner (Cyprinella bocagrande), Longnose shiner (Notropis longirostris), Mexican red shiner (Cyprinella rutila), Mimic shiner (Notropis volucellus), Mirror shiner (Notropis spectrunculus), Mountain shiner (Lythrurus lirus), Nazas shiner (Notropis nazas), New River shiner (Notropis scabriceps), Ocmulgee shiner (Cyprinella callisema), Orangefin shiner (Notropis ammophilus), Orangetail shiner (Pteronotropis merlini), Ornate shiner (Cyprinella ornata), Ouachita Mountain Shiner (Lythrurus snelsoni), Ouachita shiner (Lythrurus snelsoni), Ozark shiner (Notropis ozarcanus), Paleband shiner (Notropis albizonatus), Pallid shiner (Hyhopsis amnis), Peppered shiner (Notropis perpallidus), Phantom shiner (Notropis orca), Pinewoods shiner (Lythrurus matutinus), Plateau shiner (Cyprinella lepida), Popeye shiner (Notropis ariommus), Pretty shiner (Lythrurus bellus), Proserpine shiner (Cyprinella proserpina), Pugnose shiner (Notropis anogenus), Pygmy shiner (Notropis tropicus), Rainbow shiner (Notropis chrosomus), Red River shiner (Notropis bairdi), Red shiner (Cyprinella lutrensis), Redfin shiner (Lythrurus umbratilis), Redlip shiner (Notropis chiliticus), Redside shiner (Richardsonius balteatus), Ribbon shiner (Lythrurus fumeus), Rio Grande bluntnose shiner (Notropis simus), Rio Grande shiner (Notropis jemezanus), River shiner (Notropis blennius), Rocky shiner (Notropis suttkusi), Rosefin shiner (Lythrurus ardens), Rosyface shiner (Notropis rubellus), Rough shiner (Notropis baileyi), Roughhead Shiner (Notropis semperasper), Sabine shiner (Notropis sabinae), Saffron shiner (Notropis rubricroceus), Sailfin shiner (Notropis hypselopterus), Salado shiner (Notropis saladonis), Sand shiner (Notropis stramineus), Sandbar shiner (Notropis scepticus), Satinfin shiner (Cyprinella analostana), Scarlet shiner (Lythrurus fasciolaris), Sharpnose Shiner (Notropis oxyrhynchus), Notropis atherinoides, Notropis hudsonius, Richardsonius balteatus, Pomoxis nigromaculatus, Cymatogaster aggregata, Shiner Mauritania (Selene dorsalis), Silver shiner (Notropis photogenis), Silver shiner (Richardsonius balteatus), Silver shiner (Richardsonius balteatus), Silver shiner (Notropis photogenis), Silverband shiner (Notropis shumardi), Silverside shiner (Notropis candidus), Silverstripe shiner (Notropis stilbius), Skygazer shiner (Notropis uranoscopus), Smalleye Shiner (Notropis buccula), Soto la Marina shiner (Notropis aguirrepequenoi), Spotfin shiner (Cyprinella spiloptera), Spottail shiner (Notropis hudsonius), Steelcolor shiner (Cyprinella whipplei), Striped shiner (Luxilus chrysocephalus), Swallowtail shiner (Notropis procne), Taillight shiner (Notropis maculatus), Tallapoosa shiner (Cyprinella gibbsi), Tamaulipas shiner (Notropis braytoni), Telescope shiner (Notropis telescopus), Tennessee shiner (Notropis leuciodus), Tepehuan shiner (Cyprinella alvarezdelvillari), Texas shiner (Notropis amabilis), Topeka shiner (Notropis topeka), Tricolor shiner (Cyprinella trichroistia), Turquoise Shiner (Erimonax monachus), Warpaint shiner (Luxilus coccogenis), Warrior shiner (Lythrurus alegnotus), Wedgespot shiner (Notropis greenei), Weed shiner (Notropis texanus), White shiner (Luxilus albeolus), Whitefin shiner (Cyprinella nivea), Whitemouth shiner (Notropis alborus), Whitetail shiner (Cyprinella galactura), Yazoo shiner (Notropis rafinesquei), Yellow shiner (Cymatogaster aggregata), Yellow shiner (Notropis calientis), and Yellowfin shiner (Notropis lutipinnis).

In certain embodiments of the invention, the fishes used in the invention are in the superorder Protacanthopterygii which include the order Salmoniformes and order Osmeriformes. Non-limiting examples of fishes in this group include the smelts and galaxiids (Galaxia species). Smelts are planktivores, for example, Spirinchus species, Osmerus species, Hypomesus species, Bathylagus species, Retropinna retropinna, and European smelt (Osmerus eperlanus).

In certain embodiments of the invention, the fishes used in the invention are in the superorder Acanthopterygii which include the order Mugiliformes, Pleuronectiformes, and Perciformes. Non-limiting examples of this group are the mullets, e.g., striped grey mullet (Mugil cephalus), which include plankton feeders, detritus feeders and benthic algae feeders; the cichlids, the gobies, the gouramis, mackerels, perches, seats, whiting, snappers, groupers, barramundi, drums, wrasses, and tilapias (Oreochromis sp.). Examples of tilapias include but are not limited to nile tilapia (Oreochromis niloticus), red tilapia (O. mossambicus x O. urolepis hornorum), and mango tilapia (Sarotherodon galilaeus).

In certain embodiments of the invention, some or all species of carps, tilapias, trouts, and salmons are excluded or less preferred. When the invention is practiced in the Gulf of Mexico using one or more of the following species of fishes: Brevoortia species such as B. patronus and B. tyrannus, Hyporhamphus unifasciatus, Sardinella aurita, Adinia xenica, Diplodus holbrooki, Dorosoma petenense, Lagodon rhombodides, Microgobius gulosus, Mugil species such as Mugil cephalus, Mugil cephalus, Mugil curema, Sphoeroides species such as Sphoeroides maculatus, Sphoeroides nephelus, Sphoeroides parvus, Sphoeroides spengleri, Aluterus schoepfi, Anguilla rostrata, Arius felis, Bairdella chrysoura, Bairdeiella chrysoura, Chasmodies species such as Chasmodes saburrae and Chasmodies saburrae, Diplodus holbrooki, Heterandria formosa, Hybopsis winchelli, Ictalurus species such as Ictalurus serracantus and Ictalurus punctatus, Leiostomus xanthurus, Micropogonias undulatus, Monacanthus ciliatus, Notropis texanus, Opisthonema oglinum, Orthopristis chrysoptera, Stephanolepis hispidus, Syndous foetens, Syngnathus species such as Syngnathus scovelli, Trinectes maculatus, Archosargus probatocephalus, Carpiodes species such as C. cyprinus and C. velifer, Dorosoma cepedianum, Erimyzon species such as Erimyzon oblongus, Erimyzon sucetta, and Erimyzon tenuis, Floridichthys carpio, Microgobius gulosus, Monacanthus cilatus, Moxostoma poecilurum, and Orthopristis chrysophtera.

The shellfishes used in the methods of the invention are preferably sedentary shellfishes, such as bivalves. Depending on the environment, freshwater, brackish water, or marine shellfishes can be used. The shellfishes of the invention include but are not limited to oysters, mussels, scallops, clams, and more particularly, Crassostrea species such as C. gigas, C. virginica, C. ariakensis, C. rivularis, C. angulata, C. eradelie, C. commercialis, Saccostrea species such as S. glomerata, S. cucculata and S. commercialis, Mercenaria species such as M. mercenaria and M. campechensis, Ostrea species such as O. edulis, O. chilensis, and O. lurida, Area transversa, Panope generosa, Saxodomus nuttili, Mytilus species such as M. edulis (blue mussel), M. coruscus, M. chilensis, M. trossulus, and M. galloprovincialis (Mediterranean mussel), Aulacomya ater, Choromytilus chorus, Tapes semidecussatus, Perna species such as P. viridis, P. canaliculus, Venerupis species such as V. decussata, V. semidecussata, Sinonovacula constricta (Razor clam), Mya arenaria (soft shell clams), Spisula solidissima (surf clams), Amusium balloti, Argopecten irradians (bay scallops), Pectan species such as P. alba. P. yessonsis, P. maximus, and Chlamys species such as C. farreri, C. opercularis, C. purpuratus and C. varia.

In many embodiments of the invention, shellfishes and fishes are both cultured within the same body of eutrophic water and in certain embodiments, cultured in proximity to each other, or in the same enclosure.

### 5.4 Systems

The systems of the invention comprise a hydrocarbon production installation or a part thereof, a body of eutrophic water containing algae proximate to the hydrocarbon production installation, and a plurality of organisms that feed on algae populating the body of eutrophic water. The term “hydrocarbon production installation” refers generally to any structure that is installed offshore for the production and processing of oil and/or gas, which includes but is not limited to, various types of drilling platforms and production platforms, as well as pipelines that connect the platforms to each other, to other facilities onshore and on the seabed. In addition, the systems comprise a plurality of enclosures, and a plurality of modules and operating subsystems. The term “enclosure” encompasses any form of confinement of aquatic organisms in water by any means visible or invisible. Non-limiting examples of an enclosure include a cage, a porous container, a substrate to which shellfishes are attached, a fluidic barrier, an energy barrier, and a compartment of a hydrocarbon production installation. The modules functionally organize the management of nutrients, algae and aquatic organisms and comprise various operating subsystems that facilitate methods of the invention, e.g., fluid transport, aeration, feeding, movement of the enclosures, transfer of organisms. The aquatic organisms used in methods of the invention are contained in enclosures that are operably associated with a hydrocarbon production installation. By “operably associated” is meant that, continuously or periodically, the enclosure and its operating subsystems are mechanically, electrically, and/or fluidically connected to a hydrocarbon production installation.

The term “platform” generally encompasses hydrocarbon production installation that has a superstructure situated above water and a submerged substructure that provides support, anchorage and/or vertical mooring. Platforms are typically used for drilling, preparing water or gas for injection into an oil reservoir, processing oil and gas, cleaning the produced water for disposal into the sea, and crew accommodation. The superstructure, also referred to as the topside, comprises a plurality of decks and a plurality of modules which house drilling equipment, production equipment, safety equipment, transportation equipment, power generators, and living quarters with catering facilities. A platform of the invention comprises a variety of modules for storing nutrients, algae culturing and aquaculture, which can be modified from existing modules. Facilities for collecting algae and/or fishes from the sea, and maintaining stocks of algae and/or fishes, are particularly preferred. Wellheads are typically located on the lower deck(s). A helicopter pad, if present, is on the top deck. In shallow waters, there can be a separate quarter platform for accommodating the crew next to a production platform as a safety precaution. A bridge connects the platforms. In a preferred embodiment, the deck comprises a wind power generating means to provide electricity for the platform and its modules. Many such wind power generating means are known and can be adopted for use on the platform, see, for example U.S. Pat. No. 4,979,871, U.S. Pat. No. 7,126,235, U.S. Pat. No. 7,347,667 and U.S. Pat. No. 7,329,099.

A variety of platforms can be adapted for use in a system of the invention, for example, caissons, well protectors, fixed platforms, compliant towers, tension leg platforms, and spars. For water depths up to about 300 meters, most of the structures are well protectors and fixed platforms which are rigidly secured to the seabed. As the water depth exceeds 300 meters, the cost of a rigid structure rises sharply with increasing depth. Platforms that do not rigidly resist wind, waves and currents but compliantly resist such lateral loadings by their inertia are deployed typically at depths exceeding 300 meters. These platforms are designed to either pivot about its base on the seabed or bend over its length. The platforms are installed in water depths from 10 to 75 meters, 50 to 100 meters, 100 to 300 meters, less than 300 meters, 200 to 500 meters, more than 300 meters, more than 500 meters, 500 to 1000 meters, and more than 1000 meters.

In one embodiment of the invention, the hydrocarbon production installation is a fixed platform. There are generally two types of fixed platform, piled platform and pileless platform. A piled platform, often referred to as a steel jacket platform, rests on a pile foundation, and is by far the most common kind of offshore structure built worldwide (see e.g., U.S. Pat. No. 2,637,978; U.S. Pat. No. 2,653,451; U.S. Pat. No. 2,927,435). Almost all platforms constructed between 1947 and the mid-70's are piled platforms. Fixed platforms are economically feasible for installation in water depths up to about 500 meters. The substructure of a piled platform comprises a metal “jacket” that is secured to piles which are driven through into the seabed vertically or at an angle. Some of the legs of the jacket can comprise flotation chambers so the platform can be towed horizontally to the site (see e.g., U.S. Pat. No. 4,080,795). Drill strings, conductors, and risers are disposed in the space below the superstructure inside the jacket. Many well protectors also comprise a jacket. Pileless platform, also known as gravity-base platform, comprises a substructure that comprises a base and at least four columns that connect the base with the superstructure. The base is a large mass of steel or steel-reinforced concrete and gravity holds the platform in position. The base can comprise hollow chambers that are used for flotation as the platform is floated out to site in an upright position. The chambers can be used for ballast or storage of fluids. One or more of the columns can comprise drill strings, production risers, pumps, and piping to the cells below. The base can be surrounded by a protective skirt on the seabed.

In another embodiment of the invention, the hydrocarbon production installation is a tension leg platform that floats above the well in water depths of 200 to 1000 meters, and is held in position by weights on the seabed. The weights are connected to the superstructure by vertical steel structures, referred to as tendons. Stabilization is accomplished by buoyancy modules on the platform located near the sea surface. An example of a tension leg platform is provided in U.S. Pat. No. 4,428,702. In yet another embodiment of the invention, the hydrocarbon production installation is a compliant tower that comprises a superstructure that is supported by a slender but substantially rigid substructure, pivoted on and extending from the seabed to the surface. They are installed in water depths ranging from 400 to 900 meters. The tower substructure comprises a number of spaced legs, coupled together by braces, and terminating each with a pile. Each pile serves as a compression spring to present tension-compression couples to withstand lateral forces during rocking motion of the upper end of the tower. See, for example, U.S. Pat. No. 4,696,603. Some compliant towers comprise a plurality of catenary guy wires that extend radially outward from the sides of the superstructure to various anchor on the seabed. See, for example, U.S. Pat. No. 4,599,014; U.S. Pat. No. 4,810,135; U.S. Pat. No. 5,588,781; U.S. Pat. No. 5,642,966. In yet another embodiment of the invention, the hydrocarbon production installation is a spar that are installed in seas of at least about 500 meters depth and up to 2000 meters, to produce hydrocarbons from undersea wells, as well as to drill the wells and store produced oil. Such spars have a tall and narrow caisson extending down from the sea surface by perhaps one or two hundred meters and riser pipes that extend down from the lower portion of the caisson to the seabed. Tensioned mooring lines extend at an incline from the caisson to anchors at the seafloor. The tall and narrow caisson is subject to only moderate forces from winds, currents, and waves that cause it to drift from a position wherein it lies directly over the lower ends of the riser pipes. See, for example, U.S. Pat. No. 6,027,286.

Many of the designs of substructures are based on common engineering principles and constructed using shared or similar mechanical structures. It will be apparent to those skilled in the art that these mechanical structures and variations thereof can be similarly modified and adapted to serve the purpose of the invention. Described herein is a non-limiting example of a structure, commonly used in the construction of a platform, that can be readily adapted for use in the invention. A metal jacket forms the bulk of the substructure of a piled platform. It is also used in many well protectors (see e.g., U.S. Pat. No. 4,818,145), and variations of the design are used in columns and towers of other types of platforms. Typically, it is formed by joining tubular steel members to form a mechanically stable structure. The jacket has a frusto-tetrahedral or frusto-pyrimidal shape. Alternatively, the jacket has the shape of a prismatoid, a prism, an antiprism, a wedge, a cupola, a square cupola, a pentagonal cupola, or a truncated polyhedron. As used herein, a prismatoid is a polyhedron where all vertices lie in two parallel planes, and a prism is a prismatoid where all cross-sections parallel to the base are the same. The top end and the bottom end of a jacket are defined respectively by the upper or lower ends of upright members. The jacket may have a plurality of triangular, rectangular and/or trapezoid sides with edges that are formed by structural members. The jacket comprises a variety of structural members, mostly made with lengths of hollow steel having a circular or polygonal cross-section. In various embodiments of the invention, the jacket comprises at least 3, 4, 5, 6, 7, 8, 9, 10 or more upright members, which are welded or otherwise fastened, at various points along the length of the upright members, to a plurality of horizontal, diagonal and/or radial members in dispositions that support the weight of the superstructure and impart rigidity to the jacket against lateral displacement. The horizontal, diagonal and/or radial members are disposed to act as braces or trusses, and may traverse the space defined by the upright members. The individual upright members can be disposed at an angle to each other, or they can be parallel to one another. The upright members can be formed by splicing together a plurality of shorter members. In certain embodiments, the jacket comprises a space frame.

The top ends of the upright members are secured or fastened to elements of the superstructure that transfer the weight of the superstructure downward to the seabed through some or all of the vertical members of the jacket. For piled platforms and certain well protectors, the bottom ends of these upright members are connected to piles driven into the seabed. Within a polygonal area of the seabed geometrically defined by the bottom ends of upright members are the drilled wells which are plugged or closed by valves. The wells can be left with suitable casing in the borehole extending to some selected depth. A conductor surrounds the casing and extends downwardly into the seabed and upwardly beyond the surface of the water. Risers, drill string, conductors, and the like extending from the seabed up to the superstructure can be attached, braced, or collared, to one or more upright members for support and stability. In certain platforms, a caisson or a column is centrally disposed in the space defined by upright members (see e.g., U.S. Pat. No. 4,557,629; U.S. Pat. No. 4,687,380). The columns of pileless platform can each comprise a single upright member or a plurality of upright members fastened together and spaced from each other by horizontal and/or diagonal members.

The jacket can be constructed by combining a plurality of jacket subunits that are welded or fastened together, each jacket subunits comprising a plurality of upright members, horizontal members, radial members, and diagonal members. The jacket subunits can be combined or stacked together to form the jacket.

In certain embodiments, a plurality of guy wires extend downward from one or more edges or sides of the jacket each to a spaced anchor pile on the seabed. The guy wire forms an incline that meets the seabed at an acute angle which depends on the distance the anchor pile is removed from the base of the jacket, and the height from the seabed of the guy wire attachment on the jacket. The guy wires can be taut or catenary, and carry a part of the lateral load generated by wind, waves, and currents.

The hydrocarbon production installations useful in the invention also encompass various subsea installations, such as but not limited to wet trees, jumpers, subsea manifolds, and risers. Wet trees refer to apparatus at the top of a subsea well that comprise valves, gauges, and connection flanges, which is operated remotely from a nearby platoform. Jumpers are short length of pipes, weighing 10 to 20 tons, that connect wellheads to manifolds. Manifolds are lengths of large diameter pipe that have multiple flanges or inlet connections, and at least one for an outlet to another larger pipe or a riser. Riser are pipes connecting a tree, a pipeline, or a manifold, all on the seabed, to the upper portion or topside of a platform. Risers can take various forms, such as but not limited to, attached risers, pull tube risers, steel catenary risers, top-tensioned risers, flexible risers, and tower risers. Attached risers and pull tube risers are affixed to the outside of a platform, such as a fixed platform. The seabed end of the riser is attached to a nearby inbound flowline or outbound pipeline. Steel catenary risers, top-tensioned risers are typically connected to floating platforms, such as compliant tower, spar, and tension leg platform. Top tensioned risers comprise on the topside, flexible pipe, motion compensator and/or buoyancy modules that alleviate vertical motion, a straight riser which runs down the substructure of the platform and is attached to the seabed, and a L-shape connector that connects with the inbound and outbound piplines. Tower risers are used with deepwater platforms, where a steel tower that is at least 3000 feet tall, are anchored to the seabed in water of a depth that is similar to the height of the tower. The tower accommodates several risers and is toped with a buoyancy tank which holds the tower vertically stable. See, for example, U.S. Pat. No. 5,439,060. Risers can be used to transport fluids between two vertically disposed locations, such as from a reservoir beneath the seabed to a deck above the sea surface, separated by a height difference of about 10 to 75 meters, 50 to 100 meters, 100 to 300 meters, less than 300 meters, 200 to 500 meters, more than 300 meters, more than 500 meters, 500 to 1000 meters, and more than 1000 meters.

In various embodiments of the invention, the enclosures are formed by physical barriers operably associated with a hydrocarbon production installation. In other embodiments, the enclosures involve means for concentrating, confining, containing, holding, or directing fishes and/or shellfishes to a body of water proximate to a hydrocarbon production installation with which the enclosures are operably associated. Water can percolate or flow through an enclosure to facilitate gas exchange, removal of waste products, and replenishment of algae. Physical enclosures of the invention can be floating, submersible, or submerged. In certain embodiments, the enclosures can be raised above the water to facilitate harvesting or maintenance. A physical enclosure of the invention generally comprises side walls, a bottom wall that is proximal to the seabed, and for some designs, a top wall that is proximal to the sea surface. The flow of water through an enclosure can be generated by wind, wave, tide, current, or other natural movement of the water, or it can be caused by various means known in the art, including but not limited to the use of fluid pumps, paddles, movement of the enclosure, power-assisted aeration, etc. Optionally, the volume of flow, the flow rate, and the direction of flow either in or out or both in and out of an enclosure, can be regulated.

In one embodiment of the invention, the enclosure is a cage which comprises a meshed frame. The mesh is attached to, mounted onto, or suspended from a plurality of frame members to form walls that enclose a space for containing the aquatic organisms. The mesh can be flexible or rigid. The mesh can be made of natural and/or synthetic materials, such as cotton, bamboo, wood, reeds, nylon and other plastics (e.g., polyamide, polypropylene, polyethylene, fiberglass), and metals (e.g., galvanized steel). Many of the materials are commercially available. In a preferred embodiment, the cage for fishes is a net cage. In another embodiment, the cage is formed by panels of rigid grating mounted onto the sides of the frame. In other embodiments, the cage is a substrate to which bivalves attach themselves and the cage does not necessarily envelope the bivalves. The mesh size is selected in accordance with the dimensions of the organisms in the enclosure and the dimensions of potential predators. A half inch mesh size can be used to contain fingerlings. The cages are designed to prevent escape of the organisms, and intrusion or access by predatory organisms, such as piscivorous fishes, crustaceans, mammals (e.g., seal), and birds. The lengths, spacing, and dispositions of the frame members define the size and shape of the cage. The cage can comprise one or more closeable portals for the fish to enter or leave the cage. The portals can be located on any walls or integrally built into one or more frame members.

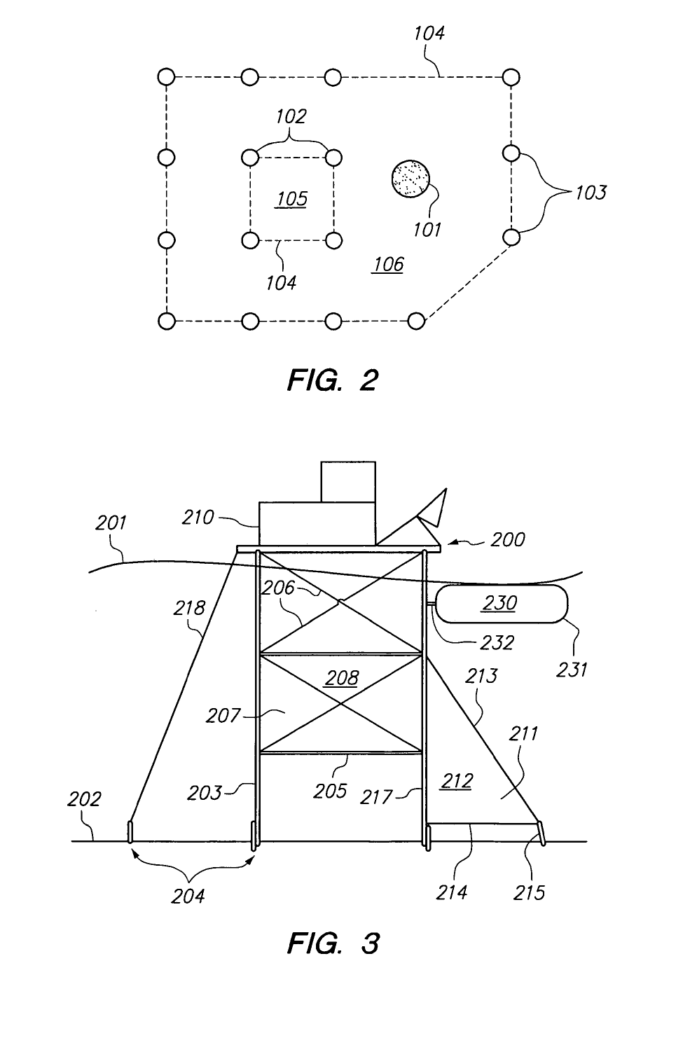

In certain embodiments of the invention, the frame of the cage 231 is rigidly or flexibly, connected by way of an attachment assembly 232 to a structure of a hydrocarbon production installation 217 at a point located at the sea surface or below, or on the seabed (see FIGS. 2 and 3). The superstructure 210 and substructure of a platform 203, 217, a tower, a spar, piles 204, 215, subsea installations, guy wires 213, 218, columns, pipes, risers 101, and conductors, can be used as attachment points.

In certain other embodiments of the invention, the structures of a hydrocarbon production installation are used as frame members, 213, 217, optionally with other frame members 214, to form the frame of a cage. See FIG. 3. Sets of nets or net panels 211 are suspended from or extended across the substructure of a platform, a tower, a spar, including any of its upright members, horizontal members, diagonal members, radial members, guy wires, columns, pipes, risers, and conductors, to define a space 212 in which the organisms are contained. Where the nets are fitted over frame members that are connected to structural members of a platform and a plurality of installations disposed a certain distance from the platform, such as a spar or subsea installations, a large enclosure can be created in the area between the platform and the spar or subsea installations.

In various embodiments of the invention, the upright members of a substructure 203, 217 divide the space under the superstructure into a plurality of vertical spaces. The division of the underwater space defined by structural members of a jacket can be used advantageously to make cages 208 for containing the organisms of the invention. For example, the vertical spaces can each be subdivided at horizontal planes, for example by extending a horizontal mesh 205, to form stacked cages. In particular, by involving variable numbers of upright members in different configurations, a plurality of vertical spaces with different polygonal cross-sections can be defined. For example, as shown in FIG. 2, the ends of a first plurality of upright members can, acting geometrically as vertices, define a polygon on a horizontal plane that encircles a second plurality of upright members. The first plurality of upright members 103 (outer or peripheral upright members) define a core-hollowed prismatoid space 106 that is circumferential to an inner prismatoid space 105 formed by the second plurality of upright members 104 (inner upright members). These prismatoid spaces can be separated by mesh 104 to form two discrete enclosures, an inner enclosure 105 and an outer enclosure 106, one inside the other. In a specific embodiment, different types of fishes are contained in the two enclosures. Preferably, planktivorous fishes are cultured in the outer enclosure and piscivorous fishes are cultured in the inner enclosure.

Floating and submersible cages (230 as shown in FIG. 3) generally require a buoyancy means, such as but not limited to a buoyant or ballastable frame member, to which a mesh is attached. Some floating cages comprise a frame member in the form of a collar that rotates about a central axis, while others rotate by moving floatation elements or by adjusting the buoyancy of the frame members. For example, a cage can be submerged through the flooding of the ballastable frame members with water pumped from a platform. Submersible cages can be kept at surface during calm weather and are lowered into the water during storms.

Many of the commercially available enclosures can be used in accordance with the methods of the invention. Minor adaptations of existing enclosures, if required, can be performed by one of skill in the art with routine experimentation. Examples of enclosures include but are not limited to flexible net cage (U.S. Pat. No. 5,299,530); rigid cages (U.S. Pat. No. 5,438,958; U.S. Pat. No. 5,628,279); cages attached or clamped directly onto the guy wires, upright, horizontal, or diagonal members of a platform (U.S. Pat. No. 5,628,279; U.S. Pat. No. 5,596,947); cages to a plurality of frame members that project outwards from the platform and are spaced to support an array of cages around the platform. See, for example, U.S. Pat. No. 5,438,958; U.S. Pat. No. 5,359,962.

The modules for managing nutrients, algae and aquaculture comprise a variety of subsystems, many of which were used in oil and gas production, and are modified to serve the purpose of the invention. These subsystems are located on one of the decks, affixed to the substructure, or connected to other platforms and subsea installations. Subsystems for fluid transport, such as oil and gas pipelines, pumps, manifolds, valves, risers, conductors, can be adapted for pumping and transporting water comprising algae and/or certain nutrients (macro- and/or micro-nutrients) from platform to platform, platform to shore, or platform to seabed, and vice versa. The cranes and derricks on the platform can be used as a subsystem for moving enclosures of the invention horizontally around the platform, or vertically from the seabed up to the surface, and even onto a deck. A non-limiting example is a winch and cable subsystem conventionally used on dredges, derrick barges and the like. For an overview of the design of cages and operating subsystems for aquaculture, see Chapters 3, 6, and 7, Cage Aquaculture. M. Beveridge, third edition, 2004, Blackwell Publishing Ltd.

Techniques for shellfish culture are well known in the art and can be adapted to the methods of the invention without undue experimentation. For example, umbrella culture uses shellfishes that have been attached to ropes and suspended from a upright member radiating to anchors like spokes on a wheel, thus taking the shape of an umbrella. Rack culture can be accomplished by adapting horizontal and traverse members of a platform (e.g., steel rebar, concrete blocks) into racks. Ropes, sticks, or nylon mesh bags with shellfishes attached or contained are placed on the racks for growout. Raft culture incorporates floating structures of a hydrocarbon production installation to suspend shellfishes off the bottom. The rafts can be made of logs, bamboo, Styrofoam, or 55-gallon drums. Raft materials are lashed together to allow flexing with wave action. Rafts are anchored to the seabed securely using subsea installations. Strings, ropes, nets (pearl nets, lantern nets), trays, and bags of shellfishes are suspended below the raft.

### 5.5 Biofuel and Fish Meal Production

Any fish processing technologies known in the art can be applied to obtain lipids from the fishes grown in and harvested from eutrophic water. For example, the fishes are subjected to gentle pressure cooking and pressing, which coagulates the protein, ruptures the fat deposits and liberates lipids and oil and physicochemically bound water; and pressing the coagulate by a continuous press with rotating helical screws. The coagulate may alternatively be centrifuged. This step removes a large fraction of the liquids (press liquor) from the mass, which comprises an oily phase and an aqueous fraction (stickwater). The separation of press liquor can be carried out by centrifugation after the liquor has been heated to 90° C. to 95° C. Separation of stickwater from oil can be carried out in vertical disc centrifuges. The lipids in the oily phase (fish oil) may be polished by treating with hot water which extracts impurities from the lipids to form biofuel. To obtain fish meal, the separated water is evaporated to form a concentrate (fish solubles) which is combined with the solid residues, and then dried to solid form (presscake). The dried material may be grinded to a desired particle size. The fish oil or a composition comprising fish lipids, can be collected and used as a biofuel, or upgraded to biodiesel or other forms of energy feedstock.

Examples of systems and methods for processing lipids such as fish lipids into biofuel, can be found in the following patent publications, the entire contents of each of which are incorporated by reference herein: U.S. Patent Publication No. 2007/0010682, entitled “Process for the Manufacture of Diesel Range Hydrocarbons;” U.S. Patent Publication No. 2007/0131579, entitled “Process for Producing a Saturated Hydrocarbon Component;” U.S. Patent Publication No. 2007/0135316, entitled “Process for Producing a Saturated Hydrocarbon Component;” U.S. Patent Publication No. 2007/0135663, entitled “Base Oil;” U.S. Patent Publication No. 2007/0135666, entitled “Process for Producing a Branched Hydrocarbon Component;” U.S. Patent Publication No. 2007/0135669, entitled “Process for Producing a Hydrocarbon Component;” and U.S. Patent Publication No. 2007/0299291, entitled “Process for the Manufacture of Base Oil.”

Preferably, the fish oil and fish meal is processed on the platform of a hydrocarbon production installation. The fish oil and biofuels obtained from fish oil can be transported ashore by the pipelines that connect the platform to shore factilities.

### 6. PREFERRED EMBODIMENTS

In a preferred embodiment, the planktivorous organism is menhaden. Menhaden is in the Clupiformes order and is representative of a large group of schooling fish that are primarily planktivores and that include but are not limited to the clupeids, sardines, anchovies, shads, herrings, pilchards, and hilsas.

Gulf menhaden Brevoortia patronus and Atlantic menhaden Brevoortia tyrannus are considered particularly suitable for use in the present invention in the Eastern shores of North America and the Gulf of Mexico, respectively. These fishes are pelagic filter feeders possessing a filtering apparatus consisting of gill rakers which strain plankton from the water and an epibranchial organ which concentrates the food accumulated on the rakers. Menhaden are commercially caught but not farmed, and the landings and wild stocks are monitored by government fishery management agency (1991, Vaughan and Merriner, Mar Fisheries Rev 53:49-57). In the wild, Atlantic menhaden abundance is correlated with abundance of microflagellates, diatoms, chlorophyll a, and shows a preference for larger phytoplankton. Prorocentrum blooms tend to correlate positively with distribution of Atlantic menhaden in North Carolina and Virginia estuarine creeks (1989, Friedland et al., Mar Ecol Prog Ser 54:1-11). Post-metamorphic juvenile Atlantic menhaden modify their distribution patterns to follow those of phytoplankton biomass distribution. Location of juvenile nurseries in North Carolina estuary is believed to be influenced by phytoplankton abundance (1996, Friedland et al., Estuaries 19:105-114). The geographic distribution of menhaden coincides with coastal areas where algal bloom and hypoxic zones occur.

The physiology and behavior of the menhaden are well known. Gulf and Atlantic menhaden have similar developmental stages: larvae at <21 mm S.L., post-larvae at 21-30 mm, juvenile at >30 mm, and young adult at >85 mm. Atlantic menhaden achieve these stages earlier than Gulf menhaden (1993, Powell, Fishery Bulletin 91:119-128). Juvenile, young adult, or adult menhaden with gill rakers that are formed after metamorphosis, are preferably used in the methods of the invention. Depending on the age of the fish, different types of phytoplankton and zooplankton with dimensions ranging from about 13 μm up to about 10 mm have been retained. Swimming speed is a behavioral response to the size and abundance of food particles present and is constant over a wide range of phytoplankton concentration (1975, Durbin and Durbin, Mar Biol. 33:265-277). A direct relationship of increased growth and larger size of juvenile Atlantic menhaden with food supply has been demonstrated in nutrient-enriched laboratory mesocosms with the highest values achieved in silicate-enhanced diatom-containing tanks. (1990, Keller et al., Limnol. Oceangr. 35:109-122). The changes in body composition and morphology of young-of-the-year gulf menhaden in Louisiana has been studied. The lipid content of gulf menhaden varied from 0 to 8%, with juveniles at 2.7% and young adults at 3.5%. The energy content of the fish relates primarily to lipid content at about 22.1 kJ/g to 27.4 kJ/g (1986, Deegan, J Fish Biol. 29:403-415). The fatty acid composition of menhaden oil have been extensively analyzed by Joseph (1985, Mar Fisheries Rev., 47:30-37).

Research-based methods for handling menhaden are known in the art. For example, methods for artificial fertilization of gulf menhaden and cross-fertilization with yellowfin menhaden (B. smithi) are reported (1968, Hettler Jr., Trans. Am. Fisheries Soc. 97:119-123). Methods for transporting adult and larval gulf menhaden and techniques for spawning in the laboratory are also reported. (1983, Hettler, The Progressive Fish Culturist, 45:45-48). It is contemplated that such husbandry and culturing techniques can be adapted with routine experimentation, for use in the practice of the invention.

Given the knowledge of the feeding behavior, bioenergetics, and lipid composition of menhaden, the inventors are using menhaden in eutrophic water for reduction of algal biomass, production of biofuels and fish meal. Methods for handling menhaden can be adapted for use with other fishes, e.g., clupeid fishes.

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

What is claimed:

1. A method for reducing algal biomass in a body of eutrophic water, said method comprising (i) providing a hydrocarbon production installation or a part thereof; (ii) populating a body of eutrophic water that is proximate to the hydrocarbon production installation with a plurality of organisms that feed on algae, and (iii) culturing the organisms that feed on algae in the eutrophic water, thereby reducing the algal biomass.

2. The method of claim 1, wherein the organisms are confined to an enclosure or a substrate operably associated with the hydrocarbon production installation.

3. The method of claim 1, wherein the body of eutrophic water comprises more than of 1×10⁵ algal cells per ml, an algal bloom, a harmful algal bloom, or a hypoxic zone; and the algal cells are cells of cyanobacteria, diatoms, dinoflagellates, raphidophytes, and/or haptophytes.

4. The method of claim 1, wherein the dimensions of at least one dominant species of algae range from about 200 to 2000 μm, 20 to 200 μm, about 2 to 20 μm, or about 0.2 to 2 μm.

5. The method of claim 1, wherein at least one dominant species in the eutrophic water is a species of Anabaena, Aphanizomenon, Microcystis, Noctiluca, Alexandrium, Prorocentrum, Gymnodinium, Ceratium, Pfiesteria, Dinophysis, Gyrodinium, Heterosigma, Chattonella, Skeletonema, Synedropsis, Pseudonitzschia, Leptocylindrus, Chaetoceros; Phaeocystis, Nannochloris, Stichococcus, Aureococcus, Miraltia, Guinardia, Spermatozopsis, Urosolenia, Nitschia, Cyclotella, Cryptomonas, or Pedinophora.

6. The method of claim 1, wherein the plurality of organisms that feed on algae comprise a population of fishes, a population of shellfishes, or populations of fishes and shellfishes.

7. The method of claim 1, wherein the plurality of organisms comprises planktivores and/or omnivores.

8. The method of claim 7, wherein at least one of the major species of planktivores is a pelagic phytoplanktivore.

9. The method of claim 1, wherein at least one major species of the organisms that feed on algae is a species of fish in the order Clupiformes, Siluriformes, or Cypriniformes.

10. The method of claim 1, wherein the plurality of organisms comprise at least one of any one of menhaden, anchovies, shiners, shads, sardines, pilchards, herring, smelts, catfish, milkfish, carps, mullets, hilsas, oysters, mussels, scallops, or clams.

11. The method of claim 1, wherein the plurality of organisms comprise Brevoortia patronus or Brevoortia tyrannus which are confined to a net cage.

12. The method of claim 1, wherein the plurality of organisms are fishes introduced at a density of 300 to 600, or 600 to 900 individuals per m².

13. The method of claim 1, wherein the step of culturing the plurality of organisms comprises replenishing the eutrophic water in the enclosure with algae, and does not comprise adding fish meal, fish oil, nitrogenous fertilizer, and/or phosphorous fertilizer into the eutrophic water.

14. The method of claim 1, wherein the step of culturing the plurality of organisms comprises maintaining the level of dissolved oxygen in the eutrophic water above 3 mg per liter.

15. The method of claim 1, said method further comprises harvesting at least one of the plurality of organisms after culturing.

16. A method for producing biofuel and/or fish meal, said method comprising (i) providing a hydrocarbon production installation or a part thereof; (ii) populating a body of eutrophic water that is proximate to the hydrocarbon production installation with a plurality of fishes that feed on algae; (iii) culturing the fishes in the eutrophic water where the fishes feed on algae in the eutrophic water; (iv) harvesting the fishes; and (v) processing the harvested fish to produce biofuel and/or fish meal.

17. A method for modifying the diversity of algal species in a body of water comprising more than 10⁵ algal cells per ml, said method comprising (i) providing a hydrocarbon production installation or a part thereof; (ii) transporting a stream of water comprising a macronutrient or a micronutrient from a distance or a different water depth relative to the body of water by the hydrocarbon production installation; and (iii) discharging the stream of water into the body of water, wherein the concentration of the macronutrient or the micronutrient in the stream of water and in the body of water is different.

18. A method for increasing the algal productivity of a body of oligotrophic water, said method comprising (i) providing a hydrocarbon production installation or a part thereof; (ii) transporting a stream of fluid comprising a macronutrient or a micronutrient from a distance or a different water depth relative to the body of oligotrophic water by the hydrocarbon production installation, and (iii) discharging the stream of fluid into the body of oligotrophic water, wherein the concentration of the macronutrient or the micronutrient in the stream of fluid and in the body of oligotrophic water is different.

19. The method of claim 18, comprising discharging a stream of fluid comprising iron, such that the concentration of iron in the body of oligotrophic water increases to a level greater than 0.1, 0.5, 1, 5, or 10 nM.

20. A system comprising a hydrocarbon production installation or a part thereof; a body of eutrophic water containing algae proximate to the hydrocarbon production installation; and a plurality of organisms that feed on the algae in the body of eutrophic water.

21. A hydrocarbon production installation-based system for regulating algal biomass in a body of water, said system comprising a nutrient management module, an algae module, and an aquaculture module, wherein said modules are operably associated with a hydrocarbon production installation.

22. The system of claim 21, wherein said nutrient management module comprises pipelines and/or risers that are fluidically connected with a plurality of hydrocarbon production installations that are horizontally spaced in a geographic area, or vertically disposed from below the seabed up to the height of a deck.

23. The system of claim 21, wherein said algae module comprises imaging subsystems for measuring algae density, counting algae, and/or classifying algae taxonomically, and data subsystems for analysis, storage, and display of algal biomass data.

24. The system of claim 21, wherein said aquaculture module comprise one or more enclosures, and a plurality of operating subsystems.

25. The system of claim 21, wherein said enclosure is a floating cage, a submersible cage, or a submerged cage.

26. The system of claim 21, wherein a plurality of structural members of the hydrocarbon production installation form parts of a frame of a cage.

27. The system of claim 21, wherein the operating subsystems comprise an environmental monitoring subsystem, a feeding subsystem, an aeration subsystem, a transfer subsystem, and a fish processing facility.
